SEQUENCE LISTING (13 sequences)

SEQ ID NO: 1 - DNA, 3216 bp, Glycine max
  CDS (291)..(2876)
gataacggctgttggaaaacaaccctttacgtttaacgtgaataggaatcaatcactattattaatttagtactttcaacttgcaactcccttgtatgtggacctttgagttccacgtatgtatacaatccatccatttgatacttgagtgctaccctcattcttattaaagggtctttttgggttgttctatgtatgcacattttcagattgcggcacttgaattaagggaaaagggttgaaacttactggtttttgttacatagtaaactaaatagtagagagcaaggatgagagttgcagtggttggtagtgggattagtgggttggcttcagcttatcttttggccaaagggggagtgaatgtggtgctctatgagaaggaggattcgttgggaggacatgccaagactgtgaatgttgatggggttgatgttgaccttggcttcatggtcttcaatcgggtcacctatcctaacatgttggatttttttgagaatcttggagttgacatggaatcatcagacatgtctttttctgttagtttagacaaaggtcgtggatgcgaatggggaagcagaaatggtttgtcaagcttgtttgcacagaagaagaatgtactgaatccttacttctggcaaatgataagggaaattgtcaagttcaaagatgatgttattagctatcttgatatgcttgagaataacccagatattgaccgcaacgagcctctaggggaattcatcaagtcaaggggttactccgaattatttcagaaagcctatcttattccaatttgtggttctatctggtcctgctcttctgaaggagttatgagtttctctgctttctcagttctttcattctgtcgcaaccaccatctacttcagctctttggcagaccacagtggctaactgtaagatggcgctcacaaacttatgttaataaggtcaaacaagagcttgagagggaaggtagtcaaataataactaatcgtgaggttcacttggtttcgacgacatctgaaaaaggatgtgttgtgtactgcaatgatggttctcaagaaatgtatgatggatgcataatggcagtgcatgcacctgatgccttgcgattattaggagatgaagcaacatatgatgagcgaagaatactcggagcttttcaatatgcttacagtgatatttttcttcatcgtgacaaaaatttaatgccccaaaacccagcagcatggagtgcatggaattttcttggtagtaacaacaacaaagtttgtctaacatactggatcaatattcttcagaatattaaagagacaagtcaaccctttcttgtaactctcaatccagaccatataccagaaaataccttgctaaagtggtcaactggccatccagtcccatcagttgctgcattcaaagcttcattagagcttgaccatattcaagggaaaagaaaaatctggttttcaggagcataccaaggttatggatttcatgaagatggatttaaggctggcatgatcgcagctcatggcattctaggaagctgctgtgcccttcagaccaacccaaaacacatggtaccttcttggaaggaactgggagcacgcatttttgtgactagattcctgagttgctatatcactactggttgtttaatgttactggaagagggaggaacaatgttcacctttgagggaactgggaaaaactgtggtctgaagagtgttttgagggtccacgatccacaattttattggaaggtgatgacacaggcagatttaggccttgcagatgcatatatcaatggggacttttcatttgttgataaagatgaaggtctcttgaatcttattttgattctcatagccaacagagattcaaatgcatccaactcaaaactgaagaagaataggggttggtggacacccgttttctttacgtctgctttgacatctgcaaagttcttcatggatcatgtttcaaggcgaaatactctcacgcaggctcgcaggaacatctctaggcattatgatctgagcaatgacctctttgcgactttcttggatgaaacaatgacatattcatgtgcagtgttcaagaataaagatgaagacttgaaagatgcacaaaagagaaaaatctctctcctgattgaaaaagctagaatagataagacacatgaaattcttgagattggatgtggatggggaagtttagccattgaagttgtcaaacaaaccggctgcaaatacactggtatcactttgtctgaggagcaactgaaacttgcagaacaaagagttaaagatgctgggcttcaggaccgcatcaattttgttctatgcgactatcgccaattgcccaagacatataaatacgataggattatatcttgtgaaatgatagaagctgttggtcatgaatacatggaagagttctttggctgttgtgaatcagtattggcggataatggacttcttgttctccagttcatatcaatcccggatgagcgttatgatgaatataggcgcagttctgattttataaaggaatatatatttccaggaggttgcctaccctctctaagtaggataacatctgccatggcagccacatccagactatgcgtggagcacgttgaaaacataggtattcattactaccaaacactcagatgctggagaaaaaacttcttgaaaaggcagaatgaaattttggctctcggattcaatgaaaaatttatcaggacatgggaatactattttgattactgtggtgcaggttttaagtcactcacactagggaattatcaggtggttttctcacgccctggtaacgttcctgcattaggcgatccatacaaaagctagaccaaaaccagttgacagaacaagcctcacacattctaccaaagtgcattacatgatggtgccaccatgtatctcctttcttgataatctcgaagtaatttttttgtcaatatagatctcttgtacattgttaaaatagaggagctgaaatgtgctgttttgccgtggcaaacagcaagattggtgagaagtttttctcacaaatgatatttttttaaaaaggaacaagcttattgatgcccaaaaacaaataactcattaaatttctgtgtaactttttatcccgtagctgacaagtaaacatataatattatccctattttgctaattttcattatct

SEQ ID NO: 2 - protein, 861 aa, Glycine max
MRVAVVGSGISGLASAYLLAKGGVNVVLYEKEDSLGGHAKTVNVDGVDVDLGFMVFNRVTYPNMLDFFENLGVDMESSDMSFSVSLDKGRGCEWGSRNGLSSLFAQKKNVLNPYFWQMIREIVKFKDDVISYLDMLENNPDIDRNEPLGEFIKSRGYSELFQKAYLIPICGSIWSCSSEGVMSFSAFSVLSFCRNHHLLQLFGRPQWLTVRWRSQTYVNKVKQELEREGSQIITNREVHLVSTTSEKGCVVYCNDGSQEMYDGCIMAVHAPDALRLLGDEATYDERRILGAFQYAYSDIFLHRDKNLMPQNPAAWSAWNFLGSNNNKVCLTYWINILQNIKETSQPFLVTLNPDHIPENTLLKWSTGHPVPSVAAFKASLELDHIQGKRKIWFSGAYQGYGFHEDGFKAGMIAAHGILGSCCALQTNPKHMVPSWKELGARIFVTRFLSCYITTGCLMLLEEGGTMFTFEGTGKNCGLKSVLRVHDPQFYWKVMTQADLGLADAYINGDFSFVDKDEGLLNLILILIANRDSNASNSKLKKNRGWWTPVFFTSALTSAKFFMDHVSRRNTLTQARRNISRHYDLSNDLFATFLDETMTYSCAVFKNKDEDLKDAQKRKISLLIEKARIDKTHEILEIGCGWGSLAIEVVKQTGCKYTGITLSEEQLKLAEQRVKDAGLQDRINFVLCDYRQLPKTYKYDRIISCEMIEAVGHEYMEEFFGCCESVLADNGLLVLQFISIPDERYDEYRRSSDFIKEYIFPGGCLPSLSRITSAMAATSRLCVEHVENIGIHYYQTLRCWRKNFLKRQNEILALGFNEKFIRTWEYYFDYCGAGFKSLTLGNYQVVFSRPGNVPALGDPYKS

SEQ ID NO: 3 - DNA, 1787 bp, Glycine max
  CDS (312)..(1448)
aaaagagcatctgcaaaagataaggagacactcactgccattcctactcttacaaaccttcttcagaacttcaactcttcgatctcctccactttgcgcatcgtactctctctctttctctctctctctcttaccttctcaactcaacatcaaaacaatatcaattctaataataactcattgcacacgtcaaatgaatagaagaagataattttgcaatatcacgtgattttgaagatgaatatcgatcgatatcgtgtccggtactactatttcagctttacaggaaatgcccatagcaatcaacactgatgagccaaaatctcaacatcgtgaattaccgattgctttcttcaagcttgtgcttggaaacaatctcaaatacagggacaatgcagcttgcatacgagactgttgtgaagctcatgctggctgcactcgaacgcaacgtgctccctgacgtcatcaccaggagactcacgcgcttgctgttggctactcgccttcgctctgcttacaaaccctcctcccaacttcaactctccgatctcctctacttcgcgcattcattacaagagatgcccatagcaatcaacactgacaagcctaagtctcaacattatgaattaccaaccgctttcttcaagctcgtcctcggaaacaatctcaaatacagctgttgttatttctcttctgcctcaatgacgctggatgatgctgaagaagcaatgttgaaactgtactgtgagagatcaaacctgaaagatggtcatacagtgcttgatgtgggatgcggttggggatcgttggctctatacattgccaagaattacactaactgtagggttacaggaatctgcaattccacaactcaaaaggcttatattgaggagaagtgtagggatcttcagctgcaaaatttgaatattatagttgctgatattagcacatttgaaatggagacttcttatgacagaatattttccatagaaatgtttgagcatatgaagaactataaagatcttctgaagaagatatccaaatggatgaaagaggatagccttttatttgttcattacttctgccacaaagcatttgcctaccactttgaggacaaaaatgaagatgactggattacaagatacttctttactggaggaactatgccttcggcaaatctacttctttatttccaagatgatgttactgtcaccaaccattggctagtaaatgggaaacactacgcacagaccagtgaagaatggctcaaaagaatggaccagagaatgactttcatcaagccaattatgcaatcaacatacggcaaggactcagctaccaagtggactgcttattggagaacattcttcatagctgtagcagaactttttggatacaataacggtgaagaatggatggttgcacacttccttttcaaaaagaaataaataagccgaaacctagtctttttatttgactatttgagaacggccttaggacaaaaaagctgctacacgaggttgcacccttcaataatgttgtatacttttattagttaacttgttttagttggttgcttgtctttatttttccctgttgtaatggtgttgccactccaaccttattttatcttaaattttgggaaagtccaattcaaccctcggaacttagggtcatgcacgattgcatgtgttctctagtccataaattttaaaatgtgcacttaagagcctttgtaacgaaaatggttatgctcaaggtagatgaccatatatttgtgcacgctc

SEQ ID NO: 4 - protein, 378 aa, Glycine max
MSQNLNIVNYRLLSSSLCLETISNTGTMQLAYETVVKLMLAALERNVLPDVITRRLTRLLLATRLRSAYKPSSQLQLSDLLYFAHSLQEMPIAINTDKPKSQHYELPTAFFKLVLGNNLKYSCCYFSSASMTLDDAEEAMLKLYCERSNLKDGHTVLDVGCGWGSLALYIAKNYTNCRVTGICNSTTQKAYIEEKCRDLQLQNLNIIVADISTFEMETSYDRIFSIEMFEHMKNYKDLLKKISKWMKEDSLLFVHYFCHKAFAYHFEDKNEDDWITRYFFTGGTMPSANLLLYFQDDVTVTNHWLVNGKHYAQTSEEWLKRMDQRMTFIKPIMQSTYGKDSATKWTAYWRTFFIAVAELFGYNNGEEWMVAHFLFKKK

SEQ ID NO: 5 - RNA, 9349 nt, Glycine max
gauaacggcuguuggaaaacaacccuuuacguuuaacgugaauaggaaucaaucacuauuauuaauuuaguacuuucaacuugcaacucccuuguauguggaccuuugaguuccacguauguauacaauccauccauuugauacuugagugcuacccucauucuuauuaaagggucuuuuuggguuguucuauguaugcacauuuucagauugcggcacuugaauuaagggaaaaggguugaaacuuacugguuuuuguuacauaguaaacuaaauaguagagagcaaggaugagaguugcagugguugguagugggauuaguggguuggcuucagcuuaucuuuuggccaaagggggagugaauguggugcucuaugagaaggaggauucguugggaggacaugccaagacugugaauguugaugggguugauguugaccuuggcuucauggucuucaaucggguaagcauugcaaugguuuucuuuuuuuucucugauucaauucuugaacacacuacuugugaggauuauuuuucuuaguuauaacuuuuuucuuacgugaagauugaauugucaauuaauccuagcuucaaaaucagucacacauuauccagaacaguaaaagggugcugcuuuuuuaaugguuuugguuuucauguaaacuuugauucucuuacauggaaugugaguguuaagaugauguuguuggaguucucaagaguguucgagauagaauuauuagaaugagaauuauguucagauuuucaaggauuaauuagaaaagauuagugaaaauaucauauucuagaagaggaauuaggaaaacucacaauuucagagacgaaaaacauugcugaauucagaaugugguaugaagccauuguuguuuuaauuccuuguaauuuuauucauuuagcaaaggaaaaguaugaacauauaccguauuuuggggugaagcugaaaauuagaaaaagauugagagaaauuugaucgauguaauaaauaacaguaaaaacguaaagagaaaaaaaaaugaaaaugaggugaaagaaaaguuagguguauuguauguuuaauaauaacauuuuagccuaaauaacuuauaaacagcugacugaccucaggagaggaauaauuaacuuaguuaauauuuugcagaauaacgaauuuauuguuuguaguguugggugaugcuuagccacaccugagcguuacuuauucuauucccauucaacagcauacauguccgguuugugagcauccauuucuccauaauugacauuuaauuucaguuuuucaaaaccuuuuuuauaguaacuccuuccaagaauuaauuaaauauuaauugcccuuuuaaaaaauuaaauauuaauuuauuagaaagaaaauugucuuuuauuuaauauugaauuuauuacuuuaguguacuuuugaaaaaauuaaauauuaauugauuaaaaaaaauuauuuaucaauagcuauaaaaauguuaucuuuuuuccaaauuuugauaauaaaaaaauaagaaaguuaauguaauuauuuauaucauuuuauauauaaauuuuuaaaauaaaaaaauaguauuuuuaaaauuaugaaaaagaauauuucaaaucaaacaaacucucccgaauaguccaugugaauguugaaucuaagaaugauuauuagaccaagagacuacaaaagccagaaaguauacgccauugcacaugaaaaggaaauauuuaguuacaauacuuuaaacauuuuauucgcccaaacacccacgugacccacguaacuauauggauuaaaaaaaaaacacucacguaacuauacuuaaguugaucaccuaacuuuacagcucuaugggccccaccuguggucaugauguuuacuuguguaguuucuaucaggcucagacucguuuggacuucuagauauugugaacccgauggacagaacgcaacuaaaaaaaugaaaaaaauugucaaauugaagcauauauauauauauauauauauauauauauauauauauauauauauauauauauauauauuuaugugcugggcauuuaauguaaccucaugugaagcaucaguauuaucaguuaaaaugggcauucaaugcuuuguuugugaaacugauugauuuggaccugacuuguugucccguacucagcuguuuagguuucuuuauaaaauuacacaauuuuaaaauugaauauuaauauaacuacaugaauagauccaguuaacaaaaggacagagaagucaauuucuauuuauuuuauaucauggaaugauuacagcagugcuugugaggcuauauauuuauugacauuguaacauggauuauugaacaggucaccuauccuaacauguuggauuuuuuugagaaucuuggaguugacauggaaucaucagacaugucuuuuucuguuaguuuagacaaaggucguggaugcgaauggggaagcagaaaugguuugucaagcuuguuugcacagaagaagaauguacugaauccuuacuucuggcaaaugauaagggaaauugucaaguucaaagaugauguuauuaggcaaguugguaccuacuauaguugaaguuaaaucuuucuuucaguguucgauugucagugaacuaagaaauaauaaaucacauaaacaucuccaaauugaugaugccuuuuguuucuaagcuuuggauaaaauuguauacagaucagcuuuaauuucucuggccugaauauugaauuugaacucaacagcaaaaaguucaaaguugugaugaacacuaauuacaaguguauaccguauaguagucuacuuccuauuguccugaacagcugaacccugaucaauuaguuggcuuucucuuuuccaauucugcaaugcagcuaucuugauaugcuugagaauaacccagauauugaccgcaacgagccucuaggggaauucaucaagucaagggguuacuccgaauuauuucagaaagccuaucuugugaguucaauggugucaccuguauuuucuauauauaucaauuuuaugcuaaguguguauauuuauaacauccaaugccuauagauuccaauuugugguucuaucugguccugcucuucugaaggaguuaugaguuucucugcuuucucaguucuuucauucugucgcaaccaccaucuacuucagguaguagcacaauugaaucuuuuccagauuuauaguggcauauaaucuaguucuauaauccaaauaacuaaagauuacuuauugauucccuugauccucucugcagcucuuuggcagaccacaguggcuaacuguaagauggcgcucacaaacuuauguuaauaaggugugauuuguugauuaucaugcaaagucuguagaccugugucacuugugaagcaguuuucauugucacucauacaagagaucuuucagcuuaagcuguaaaucauuuuuuugucauaaaaucaggucaaacaagagcuugagagggaagguagucaaauaauaacuaaucgugagguucacuugguuucgacgacaucugaaaaaggugauaaagcaauccaucuguuacuuuuucaauuccuuccucuucagaaagggaaggugaauucaauuggauugauuggcaugcacgucgcuuuuuuuucaaauaauaauuauauauagacuauguaaugcugcucuaguaacaauguuggaaaaauuauugcaggauguguuguguacugcaaugaugguucucaagaaauguaugauggaugcauaauggcagugcaugcaccugaugccuugcgauuauuaggagaugaagcaacauaugaugagcgaagaauacucggagcuuuucaauaugcuuacagguaaaaucccuucuaauauuucuaauuguuagcuacccuccucucagggaauaagauaacacaaauuguagugguuauauuuacaauaucuuuguguuaucauuuccguagacucaauagaguugacguuggauaguaaugcuuuuaucagcguguuaucaaaccaaaggaugaaaacaaucuuaaauuuauugucuuaggaccuauggcagaaauuuaaguauuauaagccuggaggcaaugguauacguuuaucucaucacauauauauuaaugacaaaaauaugugaaguugcuaaaaucaagaaucuuuuuucaucuauauucaaauaaguccaaaugcacaauuauucuuugaucuguauuggcagugauauuuuucuucaucgugacaaaaauuuaaugccccaaaacccagcagcauggagugcauggaauuuucuugguaguaacaacaacaaaguuugucuaacauacuggaucaauauucuucagguuggcaaauuuuauauaaaugcauugaucauaacugaacugucuugggccuauucuaucuaaagaggguaauggacaaaauuguugguuaauauuuauucuaauuugcagaauauuaaagagacaagucaacccuuucuuguaacucucaauccagaccauauaccagaaaauaccuugcuaaaguggucaacuggccauccagucccaucaguugcugcauucaaagcuucauuagagcuugaccauauucaagggaaaagaaaaaucugguuuucaggagcauaccaagguacauuccugcuuauuguauugacaaucaagauaaacuacuaauaaucaugacaguacuagcuuccaugaacucucucaugccuacuaaauuauguauaucuaaucuccauuuuuuuuagaaacaaccuuuguuagguugaggaauuuauauacuucaaauauaccaagugaucuaaccauauauuugugcuuuuucagguuauggauuucaugaagauggauuuaagguaguuaaagcuaaguauauaaugaaagaagaaauauaguuuuaacugaaaaauauuucuuugaagaguuuccuuaaaguggaaaugucuugaggcaauuaauuuaauuacaaauugucaccaacccguugaaaugaaacaaaacuuaacuauacgauugaacuuucaggcuggcaugaucgcagcucauggcauucuaggaagcugcugugcccuucagaccaacccaaaacacaugguaccuucuuggaaggaacugggagcacgcauuuuugugacuagauuccugaguugcuauaucacuacugguuguuuaaugugagacaucuagcaguaagaauucucucaaaaaacaaaaaaguaucauaaucauaugcuugauguuaauauaccaagauguaucaggauaguagugaacacaccuaaguacuuuggaauuauacaagcuucaguuacucuuucaaguugguaauauauucauucaauacguaugcaaguagaauuggcauucauuuagacacaggaucuuuacuuguaauuuggaaaauuguacaaagcuuaucauuuacuuguaugcucgaagaccaaaaauugaaugagggugauuccaaaaacaauagaggaucuauuggaauugcuauuuaugaaaacaauaauaaacauuuacuuugauaucaugcucuagacuguucuuagcauaauuuuuuuuuuuauuucaaagcuugacaauaacaagguccaaaauauaucaucuacuucaucuucuauggauuugacugguaaaaaaugauucaaguuaaugguacauuuaccuugagguguacagguuacuggaagagggaggaacaauguucaccuuugagggaacugggaaaaacuguggucugaagaguguuuugaggguccacgauccacaauuuuauuggaagguauauacuuuuaaauuucacaugaacuaugauuauugauucaugucauucuagcugcggaagugacaagauaugcuuaucuuugucaaugcaacucuaaaauuaaguagcuagcaaggagcuaaaacuaaucauuuguacguauauuucaggugaugacacaggcagauuuaggccuugcagaugcauauaucaauggggacuuuucauuuguugauaaagaugaaggucucuugaaucuuauuuugguaagcauucucaucugaaucuuucagugaguugucucuuugggacucuacacuucaauauaccaugguauuuuuacugcagauucucauagccaacagagauucaaaugcauccaacucaaaacugaagaagaauagguaaguggauauguaaguugaagccuacacuuacauauuaacuguaucggcauaucaaacuuuuuuuguuuuaugccuuuagggguugguggacacccguuuucuuuacgucugcuuugacaucugcaaaguucuucauggaucauguuucaaggcgaaauacucucacgcaggcucgcaggaacaucucuaggcauuaugaucugguaugagcacauuauccaacaauauaaaguagugugagcauauaacuaguuacacacaaucacaaaaaagucaaaugaaagauucuuuaucaagagagauaacucaaauauuugaagacugaguuuacucucaaaauguauguaccugucgaaggaauaaugaagaggcucggaauaggaaauaucuuccaagaacuauguuacagacuguagucaugcauaggagauccaugaagacuguagacuacauacaugaauaacauggacuggaucuuucuuuugccuugguguaacaugaauauagacauacucaaugcuucgguuacaguuuauagauugugaauagcauauauacaguuuguuauaccacaauauuucagcuagaaauauauauugcuugacaaagaaguguaauugaauuucucaauugcucucucuuuuguguuaacauuuuauuuaugcauauuccauaguuuuuugagaccugacacuaaaugaacagaagcuguaauuuuucuuuggccuguuucuagagcaaugaccucuuugcgacuuucuuggaugaaacaaugacauauucaugugcaguguucaaggcaagaacaacaacuaucugaugcuugagucagaauuucacauuauaaauauuucuccaguuucuggagcagcgcugguuaauacucuuccuucuuuuuuguuaaacuauuugguagaauaaagaugaagacuugaaagaugcacaaaagagaaaaaucucucuccugauugaaaaaguaagugccuuugauugcuaucuuauuacuaguauuuuaguuguuaucuuacaaaaggucuuauaagaacuuuccuucaauaaaucaaggcuagaauagauaagacacaugaaauucuugagauuggauguggauggggaaguuuagccauugaaguugucaaacaaaccggcugcaaauacacugguaucacuuugucugaggagcaacugaaacuugcagaacaaagaguuaaagaugcugggcuucagguauuuacaagcucauuuauuuucacaguuucacgaauuucauaacauaaaauaugauggauuuuuuuaugaaauuuggaaaagcuaaaugcaauguuagaaauucucaagugaauucauguuauuuuauucaugguuccaggaccgcaucaauuuuguucuaugcgacuaucgccaauugcccaagacauauaaauacgauaggauuauaucuuggugagcuauuuaucauuuaauuuacuuugcagacuaguugcaauuuuccaacacauugcauaacuuguauaagcauacuucagugaaaugauagaagcuguuggucaugaauacauggaagaguucuuuggcuguugugaaucaguauuggcggauaauggacuucuuguucuccagguguuugauagauuauuacuuuuauuaucuguaugguucuuuuuuauguaauguuuagauuaaaaaacuuuauacagggcaugcuauacaaaggaaacaagaacaugcacauuauuuuugguauuuaaauuacugcuaacuauugaaaagaugguuauuuucuauaguucauaucaaucccggaugagcguuaugaugaauauaggcgcaguucugauuuuauaaaggaauauauauuuccaggagguugccuacccucucuaaguaggauaacaucugccauggcagccacauccagacuaugguacaauuuacaucuaaauagaguuuuaucaaugagcuuucccuucauucuuuucucuuacagaaaagaauuuucuuaaaugcagcguggagcacguugaaaacauagguauucauuacuaccaaacacucagaugcuggagaaaaaacuucuugaaaaggcagaaguaugcuauuaauuugauugaaacucacuuuuguauguuaaauucaaguaacuacuuacaacagcuaauuauuauguuuauuuuugugcugacagugaaauuuuggcucucggauucaaugaaaaauuuaucaggacaugggaauacuauuuugauuacuguggugcagguuuuaagucacucacacuagggaauuaucagguauauuacaguuacuguuacaaucuuggaguaacuuuauccauuaaacucacuaguuaagaugacuuggcauagccuuaugugucauucuuaugugcuggccaucauccuacaagaaaguaccaaccuaaacuuagcuauuauguaauacugacaacagcaaucuagucuucucucaauggucaaagucugccacaugaauuaaaccacguuaugguaacaaaguuaguguuaauuuugcuaucauguaaucuaccaguaaaauaucauuuucuagaaaaacaacccaaauuucuguaacaaacuugauguuaauuuugcugaaauuuagguuaaccuuugaguacuugauuaacaagauauucuauucaacagugacacuugucaaaauguaacauguugcucgcaguugcgauuguaacaaaacaaguauauaauauguuuuauguaccauguaggugguuuucucacgcccugguaacguuccugcauuaggcgauccauacaaaagcuagaccaaaaccaguugacagaacaagccucacacauucuaccaaagugcauuacaugauggugccaccauguaucuccuuucuugauaaucucgaaguaauuuuuuugucaauauagaucucuuguacauuguuaaaauagaggagcugaaaugugcuguuuugccguggcaaacagcaagauuggugagaaguuuuucucacaaaugauauuuuuuuaaaaaggaacaagcuuauugaugcccaaaaacaaauaacucauuaaauuucuguguaacuuuuuaucccguagcugacaaguaaacauauaauauuaucccuauuuugcuaauuuucauuaucu

SEQ ID NO: 6 - DNA, 25 bp, Artificial
  Synthetic DNA
actatacgattgaactttcaggctt

SEQ ID NO: 7 - DNA, 22 bp, Artificial
  Synthetic DNA
gcttcctagaatgccatgagct

SEQ ID NO: 8 - DNA, 22 bp, Artificial
  Synthetic DNA
tcgtatttatatgtcttgggca

SEQ ID NO: 9 - DNA, 18 bp, Artificial
  Synthetic DNA
ggaccgcatcaattttgt

SEQ ID NO: 10 - DNA, 25 bp, Artificial
  Synthetic DNA
agaaaaatctctctcctgattgaaa

SEQ ID NO: 11 - DNA, 25 bp, Artificial
  Synthetic DNA
agtaataagatagcaatcaaaggca

SEQ ID NO: 12 - DNA, 20 bp, Artificial
  Synthetic DNA
tcatcaccaggagactcacg

SEQ ID NO: 13 - DNA, 19 bp, Artificial
  Synthetic DNA
aagttgggaggagggtttg

(19) United States
(12) Patent Application Publication
(10) Pub. No.: US 2024/0298599 A1
(43) Pub. Date: Sep. 12, 2024

(54) SOYBEANS HAVING LOW FURAN FATTY ACID CONTENT

(71) Applicants: SAGA UNIVERSITY, Saga (JP); J-OIL MILLS, Inc., Tokyo (JP)

(72) Inventors: Toyoaki ANAI, Fukuoka (JP); Satoshi WATANABE, Saga (JP); Naruto MAKITA, Tokyo (JP); Ryo OKABE, Tokyo (JP); Hisashi ARAI, Tokyo (JP); Erina HIYAMA, Tokyo (JP); Takashi SANO, Tokyo (JP)

(21) Appl. No.: 18/272,831

(22) PCT Filed: Jan. 14, 2022
(86) PCT No.: PCT/JP2022/002307

§ 371 Date: Jul. 18, 2023

(30) Foreign Application Priority Data
Jan. 21, 2021 (JP) 2021-007795

(51) Int. Cl. A01H 6/54; A01H 1/02; A23K 10/30; A23L 33/115; A61K 36/48; C12N 15/82

(52) U.S. Cl. CPC A01H 6/542; A01H 1/02; A23K 10/30; A23L 33/115; A61K 36/48; C12N 15/8247

(57) ABSTRACT

Provided are: modified soybeans having a reduced furan fatty acid content in the oils and fats contained in the soybeans; and uses of the modified soybeans. The present invention relates to modified soybeans in which the content ratio of furan fatty acid in the oils and fats contained in the soybeans has been reduced by genetic mutation.

### TECHNICAL FIELD

The present invention relates to a modified soybean having reduced furan fatty acid content in the oil/fat contained in the soybean, and the like.

### BACKGROUND ART

Soybean is a world's major source of edible oils/fats of plant origin. Since the composition of fatty acids contained in oils/fats has a direct influence on the quality of the oils/fats, modification of fatty acid compositions is a major issue in the breeding of soybeans.

In general, edible oils/fats are degraded by light, heat, and the like, and develop an odor that can cause a flavor problem. In particular, soybean oils/fats produce a peculiar odor called “light-induced odor (or “light exposure odor”)” in the presence of light or “heat-induced odor” caused by heat cooking or the like. Therefore, it has been pointed out that edible oils/fats containing a high percentage of soybean oils/fats, or food products and the like containing soybean oils/fats cannot be guaranteed to have acceptable quality (Patent literature 1). A degradation product of “furan fatty acid” in the fatty acid composition of the soybean oil/fat is thought to be a cause of these light-induced odor and heat-induced odor.

### CITATION LIST

Patent literature 1: Japanese Patent Publication No. 4290222

### SUMMARY OF INVENTION

Under these circumstances, it has been desired to develop a modified soybean having a reduced furan fatty acid content (production amount of furan fatty acid) in the oil/fat contained in the soybean.

The present invention was made in consideration of the above circumstances and provides the following modified soybeans, and the like.
(1) A modified soybean in which the content ratio of furan fatty acid in the oil/fat contained in the soybean has been reduced by genetic mutation.
(2) The soybean according to (1) above, wherein the content ratio is 0 ppm by mass or more and 20 ppm by mass or less.
(3) The soybean according to either one of (1) and (2) above, wherein the furan fatty acid is 3,4-dimethyl-5-pentyl-2-furannonanoic acid.
(4) The soybean according to any one of (1) to (3) above, which has a mutation introduced into Glyma20g34340 gene and/or Glyma04g05690 gene.
(5) The soybean according to (4) above, wherein the mutation introduced into the gene is any of the mutations selected from the group consisting of missense mutation, nonsense mutation, frameshift mutation, and null mutation.
(6) The soybean according to either one of (4) and (5) above, wherein the mutation introduced into the Glyma20g34340 gene is:
(i) a mutation of a nucleotide in the codon encoding the 410th amino acid residue of the protein encoded by the gene:
(ii) a mutation of a nucleotide in the codon encoding the 688th amino acid residue of the protein encoded by the gene; or
(iii) a mutation of a nucleotide present at the boundary between the 17th exon and intron of the gene.
(7) The soybean according to (6) above, wherein the mutation of (i) above is a mutation where a codon of glycine is replaced with a codon of aspartic acid.
(8) The soybean according to (7) above, wherein the mutation of (i) above is a mutation where the 1519th nucleotide, G (guanine), in the cDNA of the Glyma20g34340 gene is replaced with A (adenine).
(9) The soybean according to (6) above, wherein the mutation of (ii) above is a mutation where a codon of aspartic acid is replaced with a codon of asparagine.
(10) The soybean according to (9) above, wherein the mutation of (ii) above is a mutation where the 2352nd nucleotide, G (guanine), in the cDNA of the Glyma20g34340 gene is replaced with A (adenine).
(11) The soybean according to (6) above, wherein the mutation of (iii) above is a mutation where the 7180th nucleotide, G (guanine), in the mRNA precursor of the Glyma20g34340 gene is replaced with A (adenine).
(12) The soybean according to either one of (4) and (5) above, wherein the mutation introduced into the Glyma04g05690 gene is a mutation of a nucleotide in the codon encoding the 64th amino acid residue of the protein encoded by the gene.
(13) The soybean according to (12) above, wherein the mutation is a mutation where a codon of arginine is replaced with a codon of cysteine.
(14) The soybean according to (13) above, wherein the mutation is a mutation where the 483rd nucleotide, C (cytosine), in the cDNA of the Glyma04g05690 gene is replaced with T (thymine).
(15) The soybean according to any one of (1)-(14) above, which is a modified Fukuyutaka soybean.
(16) LF65 soybean whose accession number is FERM BP-22392.
(17) 16SN279 soybean whose accession number is FERM BP-22413.
(18) An ingredient for food products, feed products or pharmaceutical compositions, the ingredient comprising the soybean according to any one of (1)-(17) above or a processed product thereof.
(19) A food product, feed product or pharmaceutical composition comprising the ingredient according to (18) above.
(20) A soybean oil/fat extracted from the soybean according to any one of (1)-(17) above.
(21) A food product, feed product or pharmaceutical composition comprising the soybean oil/fat according to (20) above.
(22) A method for producing a modified soybean, the method comprising crossing the soybeans according to any one of (1)-(17) above with each other, or crossing said soybean with other soybean species.

### Advantageous Effects of Invention

The present invention can provide a modified soybean having a reduced furan fatty acid content (production amount of furan fatty acid) in the oil/fat contained in the soybean. An edible oil/fat derived from this modified soybean and food products containing this edible oil/fat are different from those containing conventional soybean oils/fats in that the unpleasant light-induced odor peculiar to soybean oils/fats is suppressed, and the heat-induced odor caused during heat cooking, etc., especially the irritating odor originating from soybean oils/fats, is suppressed. Furthermore, in the edible oil/fat derived from the modified soybean, the increases in viscosity, color tone and oxidation caused during heating are more suppressed than those in conventional soybean oils/fats. In addition, food products and the like containing the modified soybean or a processed product of said modified soybean have a good flavor with a suppressed beany smell. Therefore, the above modified soybean is highly useful and practical.

### BRIEF DESCRIPTION OF DRAWINGS

FIG. 1 Figure showing the furan fatty acid contents (F3/internal standard (IS)×1000) of Fukuyutaka (original variety) and four mutant lines (Mutants 1-4) (corresponding to the results shown in Table 1 in Example 1).

FIG. 2 Figure outlining the identification of the locus region of the mutant gene using the segregating population obtained by crossing Toyoshirome with Mutant 1.

FIG. 3 Figure showing DNA polymorphisms and nucleotide polymorphisms found in the Glyma20g34340 gene for Mutants 1-3 having a phenotype of low furan fatty acid content.

FIG. 4 Figure showing DNA polymorphism found in the Glyma04g05690 gene (Glyma04G054100 gene) for Mutant 4 having a phenotype of low furan fatty acid content.

FIG. 5A Figure showing the results of genotyping for the 42,720,119th DNA polymorphisms in soybean chromosome 20 of Mutant 1 (#65) by PCR using high melting curves.

FIG. 5B Figure showing association between the 42,720, 119th DNA polymorphisms in soybean chromosome 20 of Mutant 1 (#65) and the furan fatty acid contents.

FIG. 6A Figure showing the results of genotyping for the 42,717,581st DNA polymorphisms in soybean chromosome 20 of Mutant 2 (16SN253) by PCR using high melting curves.

FIG. 6B Figure showing association between the 42,717,581st DNA polymorphisms in soybean chromosome 20 of Mutant 2 (16SN253) and the furan fatty acid contents.

FIG. 7A Figure showing the results of genotyping for the 42,717,998th DNA polymorphisms in soybean chromosome 20 of Mutant 3 (16SN693) by PCR using high melting curves.

FIG. 7B Figure showing association between the 42,717,998th DNA polymorphisms in soybean chromosome 20 of Mutant 3 (16SN693) and the furan fatty acid contents.

FIG. 8A Figure showing the results of genotyping for the 4,340,379th DNA polymorphisms in soybean chromosome 4 of Mutant 4 (16SN279) by PCR using high melting curves.

FIG. 8B Figure showing association between the 4,340,379th DNA polymorphisms in soybean chromosome 4 of Mutant 4 (16SN279) and the furan fatty acid contents.

### DESCRIPTION OF EMBODIMENTS

Hereinafter, the present invention will be described in detail. The scope of the present invention is not bound by these descriptions, and the present invention may be implemented with suitable modifications other than those described in the following examples, provided that the purpose of the present invention is not impaired. The present specification incorporates the specification of Japanese Patent Application No. 2021-007795 (filed on Jan. 21, 2021 (Reiwa 3)) in its entirety, which serves as the basis for claiming priority of this application. In addition, all publications cited herein, such as prior art literature, patent application publications, patent publications and other patent literature, are incorporated herein by reference.

### 1. Modified Soybean

As described above, the present invention provides a modified soybean in which the content ratio of furan fatty acid in the oil/fat contained in the soybean has been reduced by genetic mutation (hereinafter sometimes referred to as “the soybean of the present invention” or “the modified soybean of the present invention”).

According to the present invention, a “soybean” refers to the whole or a part of an individual soybean plant, which includes an individual plant as well as a partial structure such as an organ (seed, etc.) of the individual plant, and a cell or cell organelle derived from the individual plant.

According to the present invention, “genetic mutation” may be, but is not limited to, a mutation by genetic recombination (modification of genetic characteristics by genetic recombination technique) or a non-genetic recombination type mutation, for example, random mutation or genome editing. Random mutation is not particularly limited and, for example, a mutation by treatment with chemicals (e.g., ethyl methane sulfonate (EMS), nitroso methyl urea, nitroso methyl urethane, etc.) or UV irradiation, a mutation introduced by employing Diversify™ PCR method, Error-prone PCR method, Rolling Circle Amplification (RCA) method, Error-prone RCA method, or any of various known random mutagenesis kits can be employed as appropriate.

The mutation introduced into the gene by any of the above methods may be, but is not limited to, any one mutation selected from the group consisting of missense mutation, nonsense mutation, frameshift mutation, and null mutation, or a combination of two or more of these mutations. A “missense mutation” refers to a mutation where a change or substitution of a nucleotide in a codon of a nucleic acid sequence changes the type of amino acid encoded by that codon. A “nonsense mutation” refers to a mutation where a change or substitution within a codon of a nucleic acid sequence changes that codon into a stop codon, so that the ORF region 3′ to the codon is no longer translated. Nonsense mutations are also called as termination mutations. A “frameshift mutation” refers to a mutation where an insertion or deletion of a nucleotide results in a shift of the codon reading frame in the region 3′ to the insertion or deletion site. Furthermore, a “null mutation” refers to a mutation where the nucleotide sequence encoding the protein is completely deleted, or where the functional protein cannot be expressed even if the nucleotide sequence is present.

According to the present invention, the soybean before the genetic mutation (the original soybean of the modified soybean of the present invention) may be a soybean variety that has not undergone any modification of the genetic characteristics by genetic recombination or the like (i.e., wild soybean), or may be a soybean variety that had undergone breeding or the like by introducing some kind of modification of the genetic characteristics into such soybean variety, where examples of such soybean variety include, but are not limited to, “Fukuyutaka”, “Toyoshirome”, “Murayutaka”, and “Bay”.

The modified soybean of the present invention is characterized in that the content ratio of furan fatty acid in the oil/fat contained in the soybean has been reduced, where this content ratio is, for example, preferably 0 ppm by mass or more and 20 ppm by mass or less, and may be 0 ppm by mass or more and 15 ppm by mass or less, 0 ppm by mass or more and 10 ppm by mass or less, 0 ppm by mass or more and 5 ppm by mass or less, 0 ppm by mass or more and 3 ppm by mass or less, 0 ppm by mass or more and 1 ppm by mass or less, and even 0 ppm by mass.

The reduced furan fatty acid in the modified soybean of the present invention is preferably, but not limited to, 3,4-dimethyl-5-pentyl-2-furannonanoic acid (also referred to as “F3”), which is represented by the following structural formula.

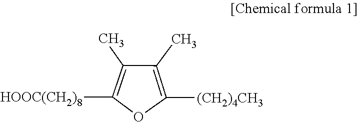

The modified soybean of the present invention is preferably one that has a mutation introduced into Glyma20g34340 gene and/or Glyma04g05690 gene (or one containing such mutation). Herein, “introduced with a mutation” or “containing a mutation” means that the mutated gene is retained in the soybean cell, and preferably the mutated gene is retained in the genome of the soybean. The mutation may also be a loss-of-function mutation in the above gene.

For details of the above Glyma20g34340 gene and Glyma04g05690 gene, for example, “Phytozome” (https://phytozome.jgi.doe.gov/pz/portal.html) in the website of “Joint Genome Institute (JGI)” (https://jgi.doe.gov/), which is one of the various publicly known databases, provides genome sequence information of the genes, cDNA nucleotide sequence information of the genes, the amino acid sequence information of the proteins encoded by the genes, and the information on the nucleotide sequence regions (CDSs) coding for the amino acid sequences.

According to the present invention, the nucleotide sequence of the cDNA of the above Glyma20g34340 gene is represented by SEQ ID NO: 1 and the amino acid sequence of the protein encoded by the Glyma20g34340 gene is represented by SEQ ID NO: 2. The nucleotide sequence of the mRNA precursor of the Glyma20g34340 gene (sequence whose representation of the genomic DNA sequence has been converted to that of RNA (thymine (T) converted to uracil (U)) is represented by SEQ ID NO:5.

Note that the gene name of the Glyma20g34340 gene is indicated as “Glyma.20G201400.1” or “Glyma.20G201400” in version Wm82.a2.v1 of the above database (Phytozome).

Meanwhile, the nucleotide sequence of the cDNA of the above Glyma04g05690 gene is represented by SEQ ID NO: 3 and the amino acid sequence of the protein encoded by the Glyma04g05690 gene is represented by SEQ ID NO: 4.

Note that the gene name of the Glyma04g05690 gene is indicated as “Glyma.04G054100.1” or “Glyma.04G054100” in version Wm82.a2.v1 of the above database (Phytozome).

Examples of the mutation introduced into the above Glyma20g34340 gene include, but are not limited to, the following genetic mutations (i)-(iii).
(i) A mutation of a nucleotide in the codon encoding the 410th amino acid residue of the protein encoded by the gene.

Here, the mutation of (i) above is preferably, for example, a mutation where a codon of glycine is replaced with a codon of aspartic acid. Specifically, it may be a mutation where the 1519th nucleotide, G (guanine), in the cDNA (SEQ ID NO: 1) of the Glyma20g34340 gene is replaced with A (adenine).
(ii) A mutation of a nucleotide in the codon encoding the 688th amino acid residue of the protein encoded by the gene.

Here, the mutation of (ii) above is preferably, for example, a mutation where a codon of aspartic acid is replaced with a codon of asparagine. Specifically, it may be a mutation where the 2352nd nucleotide, G (guanine), in the cDNA (SEQ ID NO: 1) of the Glyma20g34340 gene is replaced by A (adenine).
(iii) A mutation of a nucleotide present at the boundary between the 17th exon and intron of the gene.

Here, the mutation of (iii) above is, for example, a mutation of a nucleotide that demarcates the boundary with the intron located immediately after the 17th exon of the gene, preferably a mutation introduced into at least one or more nucleotides of “GU” (7180th-7181st) and “AG” (7267th-7268th), which designate the boundary of the intron specified by the 7180th-7268th nucleotides in the mRNA precursor of the Glyma20g34340 gene (SEQ ID NO:5) and more preferably a mutation where the 7180th nucleotide, “G” (guanine), in the mRNA precursor (SEQ ID NO:5) is replaced with “A” (adenine).

An example of the modified soybean of the present invention in which the mutation of (i) above has been introduced into the Glyma20g34340 gene include a soybean variety of the “#65” line (hereinafter also referred to as “LF65”), where this soybean variety has been deposited at the International Patent Organism Depositary, Biological Resource Center, National Institute of Technology and Evaluation (Room No. 120, 2-5-8 Kazusakamatari, Kisarazu-shi, Chiba, 292-0818, Japan) under the deposition number “FERM BP-22392”, as of Jul. 16, 2020. Examples of the modified soybean of the present invention into which the mutation of (ii) above has been introduced include a soybean variety of the “16SN253” line and examples of the modified soybean into which the mutation of (iii) above has been introduced include a soybean variety of the “16SN693” line.

Meanwhile, examples of the mutation introduced into the above Glyma04g05690 gene include, but are not limited to, a mutation of a nucleotide in the codon encoding the 64th amino acid residue of the protein encoded by the gene.

Here, this mutation may preferably be, for example, a mutation where a codon of arginine is replaced with a codon of cysteine. Specifically, the mutation may be a mutation where the 483rd nucleotide, C (cytosine), in the cDNA (SEQ ID NO:3) of the Glyma04g05690 gene is replaced with T (thymine).

An example of the modified soybean of the present invention where the above mutation has been introduced into the Glyma04g05690 gene include a soybean variety of the “16SN279” line (hereinafter also referred to as “16SN279”), where this soybean variety has been deposited at the International Patent Organism Depositary, Biological Resource Center, National Institute of Technology and Evaluation (Room No. 120, 2-5-8 Kazusakamatari, Kisarazu-shi, Chiba, 292-0818, Japan) under the deposition number “FERM BP-22413”, as of Jan. 14, 2021.

### 2. Use

The present invention provides an ingredient for food products, feed products or pharmaceutical compositions (or medicines), which contains the aforementioned modified soybean of the present invention or a processed product thereof. Also provided by the present invention is a soybean oil/fat extracted from the aforementioned soybean of the present invention. In addition, the present invention also provides a food product, a feed product or a pharmaceutical composition (or medicine) containing the above ingredient or the above soybean oil/fat.

A “processed product of soybean” refers to a soybean that has been subjected to treatments such as enzymatic treatment, powdering, drying, heating, freezing, or refinement. Specific examples of the processed product of soybean include full-fat soy flour, non-fat soy flour, and soybean protein.

“Food products” refer to products that are eaten for the purpose of nutrient intake or palatability. Soybeans have long been consumed as a food. The soybean of the present invention may be eaten as a fresh food like edamame, as a fermented food product like natto, or as a processed food product like dried beans, miso, soy sauce, bean curd, bean curd skin, soy milk, and soybean flour. While food products that can use the above-mentioned processed product of soybean as an ingredient are not limited, they may be, for example, food products in which some or all of the meat contained in processed meat products is replaced with a processed soybean product, such as hamburger steak, patties, Chinese dumplings, fried-chickens, sausages, croquettes, nuggets, shumai, pork cutlets, chicken fillets, minced meat, meat ingredients in rice products like fried rice, and meat ingredients in instant noodles, and bakery foods such as cookies, cakes, doughnuts, and breads. While the use of the soybean oil/fat is not limited, it may be used for, for example, deep frying oil, stir frying oil, rice cooking oil, and pouring oil. Examples of food products containing the soybean oil/fat include deep-fried foods, stir-fried foods, rice foods, rice crackers, noodles, seasonings such as mayonnaise and dressings, powdered oils/fats, and processed oil/fat food products such as margarine and shortening. Since the soybean of the present invention has a reduced amount of furan fatty acid in the soybean oil/fat and thus has the light-induced odor and heat-induced odor suppressed, the consumer is expected to be more comfortable for its use in foods and beverages. The soybean of the present invention may also be used as an ingredient in supplements, health functional food products, and food products for specified health uses.

“Feed products” refer to those fed to reared animals, where “reared animals” include livestock, poultry, farmed fish, and pets. Soybean is widely used in livestock and poultry feeds for cattle, pigs, chickens, and the like, and in pet foods.

“Pharmaceutical compositions” (or “medicines”) refer to those that are administered to a subject, such as a human or animal, to diagnose, treat, or prevent a disease in the subject, and may include quasi-drugs as long as such efficacy is obtained. Isoflavones contained in soybeans are known to exhibit an agonist effect on estrogen receptors and are used as a therapeutic drug for osteoporosis.

### 3. Crossing

A novel modified soybean that has the characteristics of the soybean of the present invention can be produced (bred) by crossing the soybeans (soybean species) of the present invention with each other or by crossing the soybean (soybean species) of the present invention with other soybean species. The novel modified soybean obtained in this way also has the characteristic of reduced content ratio of furan fatty acid in the oil/fat contained in the soybean, as described in section 2. above, and the like.

Preferably, the soybean species of the present invention can be crossbred with other soybean species to produce a novel modified soybean species in which a mutation (loss-of-function mutation, etc.) has been introduced into the Glyma20g34340 gene and/or Glyma04g05690 gene. For example, by crossing the soybean species of the present invention with other soybean species that are tolerant to certain pests and/or growing conditions, a novel soybean species can be produced that has a reduced furan fatty acid content in the oil/fat contained in the soybean and that is tolerant to certain pests and/or growing conditions.

According to the present invention, “crossing” refers to a crossing between two individuals with different genetic compositions, which results in the formation of a hybrid. Backcrossing is preferred as the method of crossing. In order to achieve a reduced furan fatty acid content in the novel soybean species obtained by crossing, it is necessary to backcross the novel soybean species with the soybean species of the present invention to obtain a homozygote of the Glyma20g34340 gene mutation or the Glyma04g05690 gene mutation. “Backcrossing” is a method of crossing an offspring of a mutant parent A and a non-mutant parent B (existing soybean variety such as Fukuyutaka) with the parent B (existing soybean variety such as Fukuyutaka) to obtain an offspring that retains the mutated gene and that has the characteristics of the parent B.

### 4. Screening Method

The soybean of the present invention preferably has a mutation in the Glyma20g34340 gene and/or Glyma04g05690 gene, so by detecting the mutation in either of these genes (or both genes), soybeans can be screened for those with reduced furan fatty acid content in the oil/fat contained in the soybean.

A method for detecting the mutated gene may be a method comprising the steps of:
(a) performing amplification treatment and/or hybridization treatment for the Glyma20g34340 gene and/or Glyma04g05690 gene of the tested soybean, using an oligonucleotide probe containing the nucleotide sequence of the mutated site of the Glyma20g34340 gene and/or Glyma04g05690 gene of the modified soybean of the present invention, and/or oligonucleotide primers prepared such that the mutated site is included in the amplified fragment when at least one of the nucleotide sequence of said gene and its complementary sequence is amplified; and
(b) detecting the mutated site by analyzing the treated product.

Specific examples of such detection method include, but are not limited to, known methods such as PCR method, TaqMan PCR method, sequencing method, and microarray method, and especially Invader assay and TILLING method when the loss-of-function mutation is a single nucleotide polymorphism (SNP).

In the case of the PCR method, it is preferable to create primers such that the 3′ end portion has a sequence complementary to the nucleotide sequence of the mutation site. By using the primers designed as such, if a sample template has a mutation, the primers will completely hybridize to the template and the polymerase elongation reaction will proceed, but if the template does not have a mutation, elongation reaction does not occur because of the mismatch of the nucleotides at the 3′ end of the primers with the template. Therefore, when PCR amplification is performed using such primers, and the amplified product is analyzed by agarose gel electrophoresis or the like, it can be judged that the sample template has a mutation if an amplified product of a predetermined size can be confirmed whereas the template does not have a mutation if the amplified product is not present. For PCR and agarose gel electrophoresis, see below: Sambrook, Fritsch and Maniatis, “Molecular Cloning: A Laboratory Manual,” 2nd Edition (1989), Cold Spring Harbor Laboratory Press.

The TaqMan PCR method is a method that utilizes a fluorescently labeled allele-specific oligo and PCR reaction by Taq DNA polymerase (Livak, K. J. Genet. Anal.

The sequencing method is a method of analyzing the presence or absence of a mutation by amplifying the region containing the mutation by PCR and sequencing the DNA sequence using Dye Terminator or the like (Sambrook, Fritsch and Maniatis, “Molecular Cloning: A Laboratory Manual” 2nd Edition (1989), Cold Spring Harbor Laboratory Press).

The DNA microarray has one end of nucleotide probes fixed on a support in an array, which includes a DNA chip, Gene chip, microchip, bead array, and else. Examples of the DNA microarray assay using DNA chips or the like include GeneChip assay (Affymetrix: see U.S. Pat. Nos. 6,045,996, 5,925,525, and 5,858,659). The GeneChip technology utilizes a miniaturized high-density microarray of oligonucleotide probes attached to a chip.

The Invader assay is a method that combines hybridization of two types of reporter probes and one Invader probe specific for each allele of a genetic polymorphism (e.g., SNP) to template DNA, with cleavage of DNA with Cleavase enzyme which has a specific endonuclease activity of recognizing and cleaving the DNA structure (Livak, K., J. Biomol. Eng. 14, 143-149 (1999): Morris T. et al, J. Clin. Microbiol. 34, 2933 (1996): Lyamichev, V. et al., Science, 260, 778-783 (1993), etc.).

The TILLING (Targeting Induced Local Lesions IN Genomes) method is a method of screening for a mutant mismatch in genomes of a mutagenized population by PCR amplification and CEL I nuclease treatment. CEL I nuclease is an endonuclease that specifically cleaves the mismatch portion of double-stranded DNA. The above PCR product will have a mismatch portion if the sample contains a mutation in the Glyma20g34340 gene and/or Glyma04g05690 gene, but will not have a mismatch portion if no mutation is contained. Thus, if the mutation is present, the mismatched pair site in the PCR product will be cleaved, and if the mutation is not present, the PCR product will not be cleaved. Therefore, the presence or absence of the mutation can be easily confirmed by analyzing the CEL I nuclease-treated PCR product by agarose electrophoresis or the like and comparing the lengths of the nucleic acid sequences. For more information on CEL I nuclease, the following literature is available: Oleykowski et al., Nucleic Acids Research, vol. 26, No. 20, 4597-4602 (1998).

In the mutation detection methods exemplified above, an oligonucleotide prepared to contain a mutated site in the Glyma20g34340 gene and/or Glyma04g05690 gene is used as the probe or primer. Thus, the present invention also provides an oligonucleotide prepared to contain a mutated site in the Glyma20g34340 gene and/or Glyma04g05690 gene.

In a case where a mutation in the Glyma20g34340 gene and/or Glyma04g05690 gene is detected by Invader assay, the primer or probe used is designed such that the SNP site is located at the 3′ or 5′ end of the nucleotide sequence of the primer or probe, at the 3′ or 5′ end of the complementary sequence, or within 4 nucleotides, preferably within 2 nucleotides, of the 3′ or 5′ end of the former two sequences (i.e., the primer or probe, or complementary sequence). Alternatively, the primer or probe is designed such that SNP is located in the middle of the entire length of the nucleotide sequence of the oligonucleotide. The “middle” refers to the region in the middle part where the number of nucleotides toward the 5′ end and the number of nucleotides toward the 3′ end from the nucleotide of the SNP are approximately equal, and if the oligonucleotide has an odd number of nucleotides, the “middle” preferably refers to five nucleotides in the middle part, more preferably three nucleotides in the middle part, and still more preferably one nucleotide exactly in the middle. Moreover, if the oligonucleotide has an even number of nucleotides, the “middle” preferably refers to four nucleotides in the middle part, and more preferably two nucleotides in the middle part.

Also, when the oligonucleotide of the present invention is used as an allele-specific probe in the Invader assay, the oligonucleotide is preferably composed of a fragment that hybridizes to the Glyma20g34340 gene and/or Glyma04g05690 gene containing a mutated site or to the complementary sequence thereof, and a fragment (flap portion) that does not hybridize to said genes or complementary sequence, which are attached to each other via the gene sequence of the mutated site or the complementary sequence thereof.

When the oligonucleotide of the present invention is to be hybridized to the nucleic acid molecules in the sample, the hybridization reaction is performed under stringent conditions.

The nucleotide sequence of the oligonucleotide of the present invention is preferably designed to have a length of at least 10 bases, more preferably 10-200 bases, still more preferably 15-150 bases, and most preferably 18-80 bases. This oligonucleotide sequence can be used as a probe for detecting the test gene and can be used as either forward (sense) primer or reverse (antisense) primer.

The oligonucleotide primer or oligonucleotide probe designed as described above can be chemically synthesized by known means/method, and it is generally synthesized using a commercially available chemical synthesizer.

The probe can be labeled with a fluorescent label (e.g., FITC, FAM, VIC, Redmond Dye, etc.) and a quencher for the fluorescent label in advance to automate the process.

### 5. Microarray

A microarray can be prepared by fixing one end of the oligonucleotide of the present invention described above to a support such as glass, silicon, or gel. An array of oligonucleotides is produced, for example, by photoirradiation chemical synthesis (Affymetrix), which employs a combination of solid-phase chemical synthesis with photolithographic manufacturing technique used in the semiconductor industry. By utilizing a photolithographic mask to clearly define the boundaries of the chemical reaction sites on the chip and by performing a specific chemical synthesis process, a high-density array with oligonucleotide probes attached at predetermined locations on the array can be constructed.

### 6. Kit

In another aspect of the invention, a kit for detecting a mutation in the Glyma20g34340 gene and/or Glyma04g05690 gene is provided, which comprises the oligonucleotide of the present invention and/or a microarray produced using said oligonucleotide. In addition to the oligonucleotide of the present invention or the microarray prepared using said oligonucleotide, such kit may also include a solution for detection reaction, an oligonucleotide as a control, a container used for the detection reaction, and an instruction manual.

Hereinafter, the present invention will be described in further detail by way of examples, although these examples are intended to illustrate the present invention and are not intended to limit the present invention.

### Example 1

### <Generation of Low-Furan Fatty Acid Soybean Mutant Line>

### 1. Plant Materials

Soybean (Glycine max (L.) Merr.) varieties used as the material were as follows.
(i) Fukuyutaka
(ii) Toyoshirome

The soybean variety Fukuyutaka was used to generate a mutant population. M1 seeds were obtained by soaking soybean seeds (M0 seeds) in an aqueous 0.3% ethyl methane sulfonate (EMS) solution as a mutagen overnight and then rinsing them under running water for 8 hours. The M1 seeds were then sown in a field to raise M1 individuals according to a normal method. For those plants that retained fertility, one M2 seed per individual was collected and stored. The following year, the stored M2 seeds were sown again in the field to raise M2 individuals. Two thousand eight hundred thirty-one lines of M3 seeds, each individually collected from each M2 individual, were used to select mutant lines with low furan fatty acid content. Toyoshirome and Fukuyutaka were separately crossed with the mutant lines according to the procedure described below to generate segregating populations. Note that “M0” above represents the original variety prior to the mutation, “M1” is the first generation after the mutation, and subsequent generations follow as “M2”, “M3”, and so on.

### 2. Method of Analyzing Furan Fatty Acid

100-500 mg of soybean seed powder was weighed, 5 ml of hexane was added, stirred vigorously, and kept warm on an aluminum block (TAITEC: DTC-1CN) at 60° C. for 30 minutes. Next, the resultant was sonicated in an ultrasonic cleaner (SHARP: UT-204) for 30 minutes, and then filtered using a qualitative filter paper (Whatman: No. 1 ϕ82 mm) to collect the filtrate. 5 ml of hexane was again added to the residue to repeat stirring, warming at 60° C., and extraction with the ultrasonic cleaner to collect the filtrate again. Air was blown for about 30 minutes using a gas spray unit (TAITEC: EN1-25) to completely volatilize hexane contained in the filtrate. The oil/fat in the centrifuge tube was collected and used for methyl esterification of the soybean oil/fat.

4 mg of soybean oil/fat was weighed, to which 600 μl of 0.2 M KOH-MeOH was added and mixed. 80 μl of 2 M HCl was added and mixed for 1 minute, and then 600 μl of a hexane solution containing an internal standard (methyl-d₃ behenate, concentration 10 μg/ml) was added and mixed for 2 minutes. After centrifugation at 13,000 rpm for 3 minutes, 500 μl of the upper layer was transferred to a new 1.5 ml tube. To this tube, 500 μl of sterile water was added, mixed for 1 minute, centrifuged at 13,000 rpm for 3 minutes, and then 100 μl of the upper layer was transferred to a glass vial and used as an analysis sample in a gas chromatograph-mass spectrometer (Agilent Technologies 5977A).

1 μL of the prepared sample was injected into a gas chromatograph-mass spectrometer in splitless mode for analysis. The analysis conditions for the equipment were as follows: helium gas was used as carrier gas at a flow rate of approximately 1 ml/min, HP-88 (Agilent: φ0.25 mm×60 m) column was used, analysis time was set to 20 min, inlet temperature was set to 250° C., temperature of the mass spectrometer connection unit was set to 230° C., ion source temperature was set to 230° C., and quadrupole mass filter temperature was set to 150° C. The temperature conditions for the column oven during the analysis period were as follows: the temperature was held at 100° C. for 1 minute, then the temperature was raised to 200° C. over 5 minutes at 20° C./min, then raised to 240° C. over 4 minutes at 10° C./min, and held at 240° C. for 4 minutes. The temperature was then raised to 250° C. over 1 minute at 10° C./min and held there for 5 minutes.

The peak areas of the internal standard (IS: molecular weight 357) and furan fatty acid (F3 (3,4-dimethyl-5-pentyl-2-furannonanoic acid): molecular weight 336) were determined from the obtained chromatogram and mass spectral data, and the ratio thereof was used as phenotypic data.

### 3. Screening for Mutants:

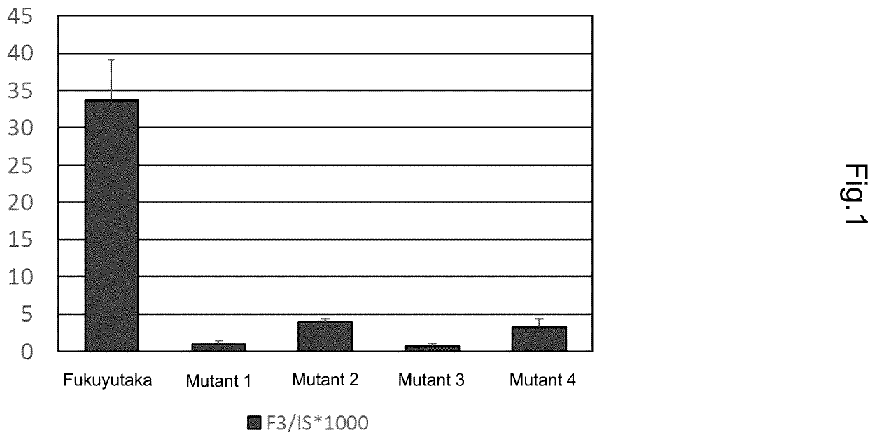

From the mutant population of the 2831 lines generated with the soybean variety Fukuyutaka, 4 lines (Mutants 1-4) were identified as mutant lines associated with the production amount of furan fatty acid by analyzing the furan fatty acid in each line using the above technique. Repeated experiments using seeds derived from different individuals confirmed that the mutations in the 4 identified lines were genetically fixed (Table A, FIG. 1).

TABLE A
Furan fatty acid content in Fukuyutaka (original variety)
and mutant lines (three times repeated measurements)

| | F3 peak area/Internal | |
|---|---|---|
| | standard peak | Standard |
| Line | area × 1,000 | deviation |
| Fukuyutaka | 33.7 | 5.39 |
| Mutant 1 | 1.0 | 0.38 |
| Mutant 2 | 4.0 | 0.37 |
| Mutant 3 | 0.8 | 0.32 |
| Mutant 4 | 3.3 | 1.04 |

4. Identification of Chromosomal Region where Mutant Gene is Located

Genetic analysis was performed to identify the mutant gene in the mutant lines selected from the soybean mutant populations. Mutant 1 and Toyoshirome were crossed through artificial selection to obtain hybrid seeds. Crossing was done in the morning on a sunny day. Toyoshirome flower from the day before flowering was used as the mother and pollen from the flower of Mutant 1 on the day of flowering was used as the father. Specifically, for the flower of Toyoshirome on the day before flowering, the calyx and petals (banner, wing, and keel) were removed with tweezers, and after visually confirming that the anthers had not split open, the anthers were removed and the stigma of the pistil was exposed. The stamen and stigma were removed with tweezers from the flower of Mutant 1 that had flowered that day, and after visually confirming that the anthers had split open, the tip of the pistil of prepared mother (Toyoshirome) was lightly touched with the tip of the stamen (pollen) to pollinate it. The resulting hybrid individual (F1 individual) was grown, and self-fertilized seeds were obtained, which were used as a segregating population (F2 population) to raise 82 individuals.

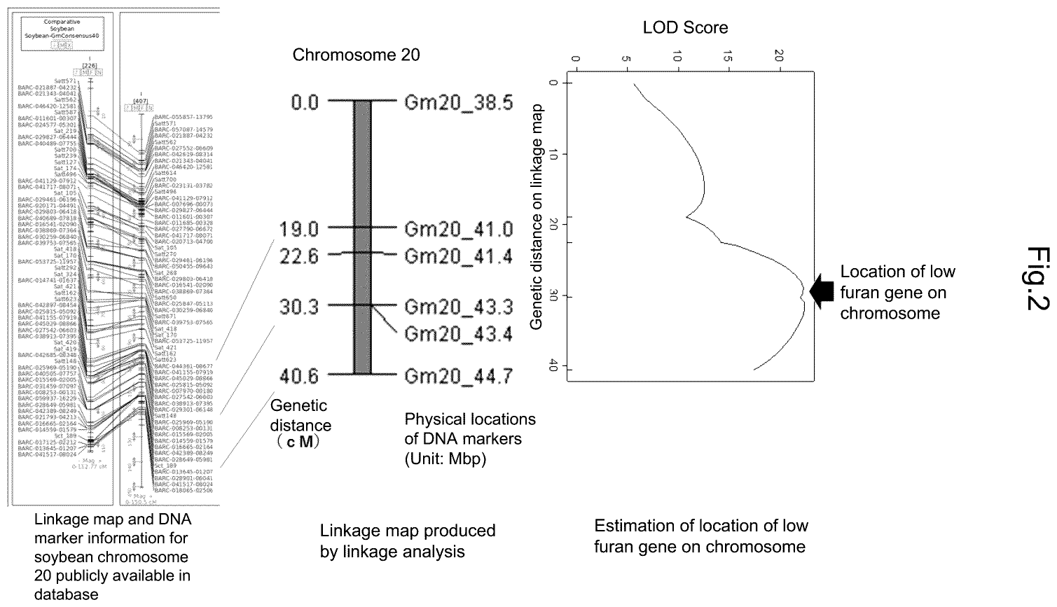

DNA was extracted from the leaves of the 82 individuals of the segregating population obtained by crossing Toyoshirome and Mutant 1 using the method described in Yamagata, Y. et al, Breed. Sci., 2018 (hereafter, Reference A). The DNA markers indicating polymorphisms between Toyoshirome and Fukuyutaka and the genotyping technique described in Reference A were employed to determine the genotype of each individual. The self-fertilized seed of each F2 individual was also used to analyze the furan fatty acid content using the method described above. Based on the genotypic information and phenotypic data of furan fatty acid content of each individual, the chromosomal region most closely related to the genotype and phenotype of each individual was identified using the statistical analysis technique described in Md. Abdur Rauf Sarkar et al., Breed. Sci., 2020 (hereinafter, Reference B). The results of the genetic analysis showed high statistics score (LOD score) for the low furan fatty acid content characteristic of Mutant 1 in the region from 41,000,000 (41M) to 45,000,000 (45M) nucleotides of soybean chromosome 20, with the highest statistic score existing around 43.3 Mbp. Thus, we located the mutated gene responsible for the low furan fatty acid content within this region (FIG. 2). The details of References A and B mentioned above are as follows.
Reference A: Yamagata, Y., A. Yoshimura, T. Anai, and S. Watanabe, Selection criteria for SNP loci to maximize robustness of high-resolution melting analysis for plant breeding. Breed. Sci. 68: 488-498, 2018.
Reference B: Md. Abdur Rauf Sarkar, Wakana Otsu, Akihiro Suzuki, Fumio Hashimoto, Toyoaki Anai, Satoshi Watanabe, Single-base deletion in GmCHR5 increases the genistein-to-daidzein ratio in soybean seed. Breed. Sci. 70: 265-276, 2020.

### 5. Identification of DNA Polymorphisms Responsible for Reduced Furan Fatty Acid Content

Whole genome nucleotide sequencing was performed for Mutants 1-4, Fukuyutaka, and Toyoshirome by next-generation sequencing analysis. DNA extracted from the leaves of each individual was sent to Novogene's NGS contract analysis service to acquire genomic information of 17 billion nucleotides per line. The genomic information of soybean (Gmax 189 hardmasked.fa) was acquired from public database “Phytozome” (https://phytozome.jgi.doe.gov/pz/portal.html) in the website of the “Joint Genome Institute (JGI)” (https://jgi.doe.gov/), and the nucleotide sequence information obtained from the next generation sequencing analysis of Mutants 1-4, Fukuyutaka, and Toyoshirome was compared with the genome sequence of the soybean using the method described in Reference B above to identify nucleotide polymorphisms (mutations) specific to the EMS-treated mutant lines (Mutants 1-4).

The results showed that Mutants 1-3 commonly had a nucleotide polymorphism (mutation) caused by mutagenic treatment in the Glyma20g34340 gene (gene name is indicated as Glyma.20G201400.1 or Glyma.20G201400 in version Wm82.a2.v1 of the database (Phytozome)) (Cyclopropane fatty acyl phospholipid synthase) located between 41M and 45M base pairs in soybean chromosome 20.

Specifically, Mutant 1 (also referred to as “#65”) had a mutation where the 42,720,119th nucleotides in the soybean chromosome 20 sequence had been replaced from C to T (i.e., a mutation where the 1519th nucleotide, G (guanine), in the cDNA (SEQ ID NO: 1) of the Glyma20g34340 gene had been replaced by A (adenine); note that since the direction of this cDNA sequence is opposite (reverse) to that of the chromosomal sequence, the substituted nucleotide in the chromosomal sequence and the substituted nucleotide in the cDNA sequence are complementary), and the 410th amino acid in the amino acid sequence (SEQ ID NO:2) encoded by Cyclopropane fatty acyl phospholipid synthase gene had changed from glycine to aspartic acid.

Mutant 2 (also referred to as “16SN253”) had a mutation where the 42,717,581st nucleotides in the soybean chromosome 20 sequence had been replaced from C to T (i.e., a mutation where the 2352nd nucleotide, G (guanine), in the cDNA (SEQ ID NO: 1) of the Glyma20g34340 gene had been replaced by A (adenine): note that since the direction of this cDNA sequence is opposite (reverse) to that of the chromosomal sequence, the substituted nucleotide in the chromosomal sequence and the substituted nucleotide in the cDNA sequence are complementary), and the 688th amino acid in the amino acid sequence (SEQ ID NO:2) encoded by Cyclopropane fatty acyl phospholipid synthase gene had changed from aspartic acid to asparagine.

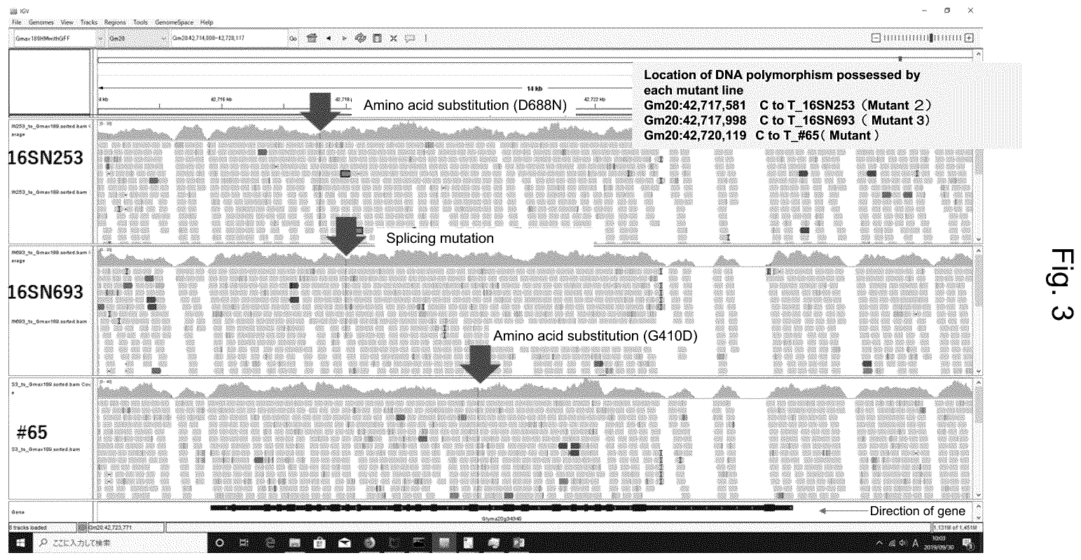

Mutant 3 (also referred to as “16SN693”) had a mutation where the 42,717,998th nucleotides in the soybean chromosome 20 sequence had been replaced from C to T (i.e., a mutation where the 7180th nucleotide, G (guanine), in the mRNA precursor sequence (SEQ ID NO:5) of the Glyma20g34340 gene had been replaced by A (adenine): note that since the direction of this mRNA precursor sequence is opposite (reverse) to that of the chromosomal sequence, the substituted nucleotide in the chromosomal sequence and the substituted nucleotide in the mRNA precursor sequence are complementary). As a result, sequence GU-AG, that was located on the border of the intron immediately after the 17th exon of the Cyclopropane fatty acyl phospholipid synthase gene (at both ends of said intron) and that was required for gene splicing (specifically, sequence that had “GU” at positions 7180-7181 and “AG” at positions 7267-7268 in the mRNA precursor sequence (SEQ ID NO:5) and that was required for splicing the intron (serving as a mark for splicing)) was changed to sequence AU-AG, and this was considered to cause splicing abnormality in the above intron (sequence identified by the 7180th-7268th nucleotides in SEQ ID NO:5) (FIG. 3).

For Mutant 4, since the Glyma20g34340 gene did not have the mutation caused by EMS treatment, the nucleotide sequence of the Glyma04g05690 gene listed as a homologous gene of the Glyma20g34340 gene in the database (Phytozome) (gene name is Glyma.04G054100 in version Wm82.a2.v1 of the database (Phytozome)) was used for the comparison.

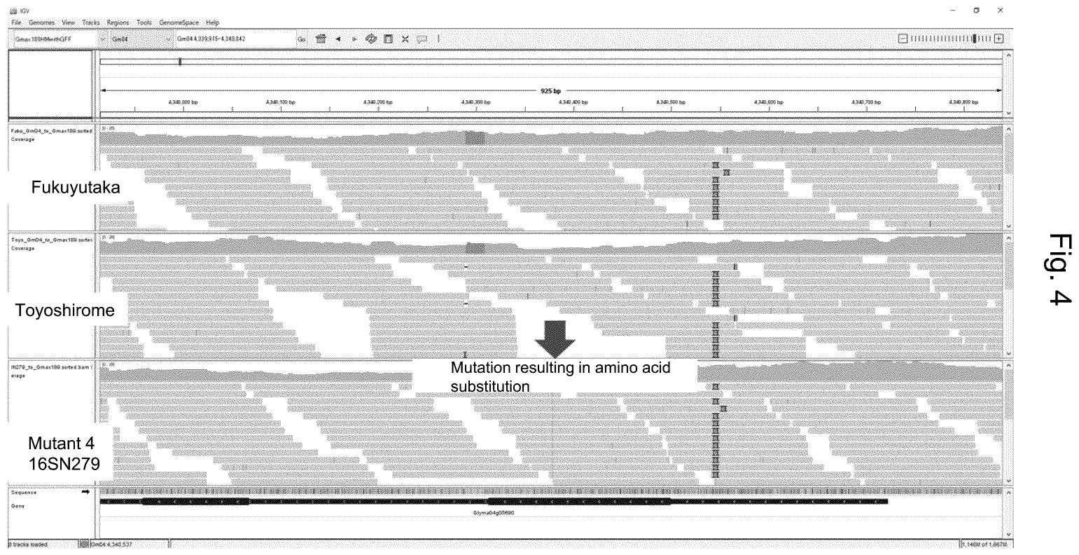

As a result, Mutant 4 (also referred to as “16SN279”) had a mutation where the 4,340,379th nucleotides in the soybean chromosome 4 sequence had been replaced from G (guanine) to A (adenine) (i.e., a mutation where the 483rd nucleotide, C (cytosine), in the cDNA (SEQ ID NO:3) of the Glyma04g05690 gene had been replaced by T (thymine); note that the substituted nucleotide in the chromosomal sequence and the substituted nucleotide in the cDNA sequence are complementary), and the 64th amino acid in the amino acid sequence (SEQ ID NO:4) encoded by the Glyma04g05690 gene had changed from arginine to cysteine (FIG. 4). The 102nd through 377th amino acids of the Glyma04g05690 gene were predicted to be involved in the function of Cyclopropane fatty-acyl-phospholipid synthase and related methyltransferases, and the 559th through 848th amino acids of Glyma20g34340 were predicted to have similar function.

6. Development of DNA Markers for DNA Polymorphisms Responsible for Reduced Furan Fatty Acid Content and their Association with Low-Furan Fatty Acid Content Phenotype

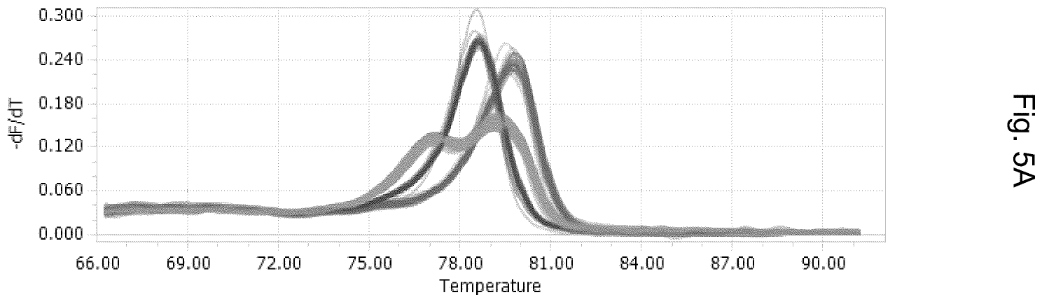

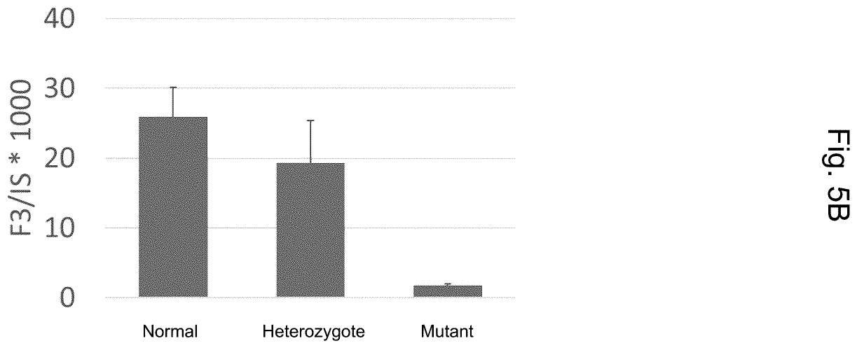

DNA markers were developed for the DNA polymorphisms identified in each mutant line (Mutants 1-4) that could identify the genotype carried by each individual, and their association with the low-furan fatty acid content phenotype was confirmed. To develop DNA markers that could identify the genotypes by PCR method for DNA polymorphisms, the method described in Reference A was employed. Specifically, primers used for PCR (forward primer: ACTATACGATTGAACTTTCAGGCTT (SEQ ID NO:6), reverse primer: GCTTCCTAGAATGCCATGAGCT (SEQ ID NO:7)) were designed to sandwich the 42,720,119th nucleotides of Mutant 1 and DNA fragments containing polymorphic DNAs were amplified by PCR method using DNA of each individual as a template. The genotype of each individual was identified by detecting the difference in the nucleotides contained in the PCR fragments as the difference in the temperature at which the double-stranded DNA melts to single-stranded DNA (FIG. 5A). Since the normal type has nucleotide C and the mutant type has nucleotide T, the peak of change in the melting temperature (hereinafter referred to as melting temperature) shows a higher melting temperature for the normal type (blue plot in FIG. 5A) than for the mutant type (red plot in FIG. 5A). On the other hand, heterozygous individuals have both normal and mutant type DNAs, so the melting temperature of the PCR fragment shows a different pattern (orange plots in FIG. 5A). The furan fatty acid content exhibited by each individual in the segregating population resulting from the crossing between Toyoshirome and Mutant 1 described above could be clearly distinguished by the genotype of the DNA marker produced. Thus, the low-furan fatty acid content phenotype can be explained by the 42,720,119th nucleotide polymorphisms in Mutant 1 (FIG. 5B).

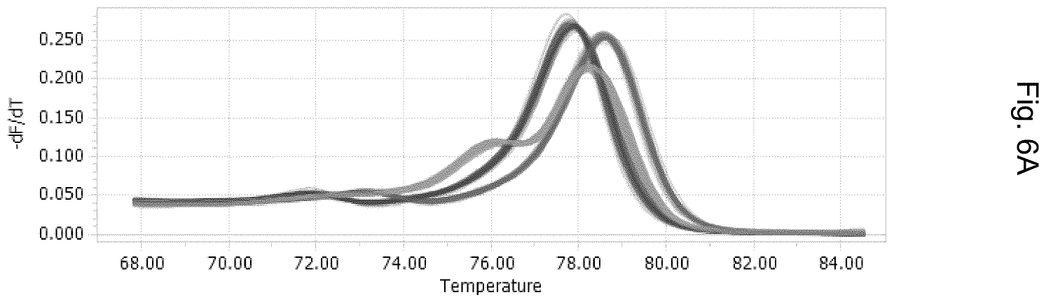

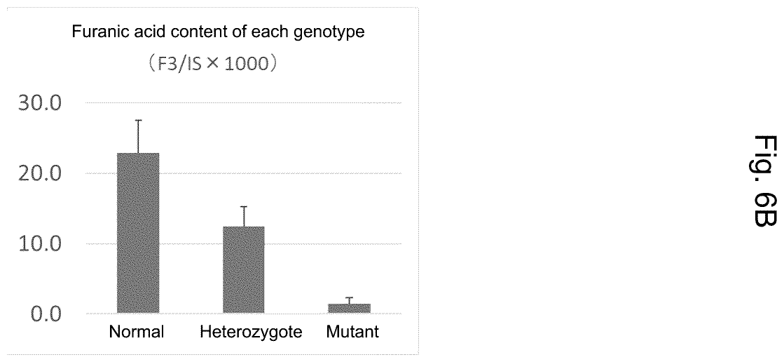

Mutant 2 (also referred to as “16SN253”) has a mutation where the 42,717,581st nucleotides of soybean chromosome 20 are replaced from C to T. Primers targeting this mutation (forward primer: TCGTATTTATATGTCTTGGGCA (SEQ ID NO:8) and reverse primer: GGACCGCATCAATTTTGT (SEQ ID NO:9)) were designed and PCR fragments containing the DNA polymorphisms of this mutant line were amplified by PCR to detect the difference in the nucleotides contained in the PCR fragments as difference in the melting temperature of the PCR fragments as described above. The normal type has nucleotide C, which shows a higher peak melting temperature than nucleotide T in the mutant type (FIG. 6A). This DNA marker was used to study the relationship between the genotypes and the furan fatty acid contents for 64 individuals from the F3 population produced by allowing the hybrid individuals obtained by crossing Fukuyutaka with Mutant 2 to undergo self-fertilization twice using the same procedure as described above. As a result, individuals with the mutant genotype expressed low-furan fatty acid content phenotype. Thus, the low-furan fatty acid content phenotype can be explained by this DNA polymorphism (FIG. 6B). The explanations for the blue, red, and orange plots in FIG. 5A also apply to those in FIG. 6A.

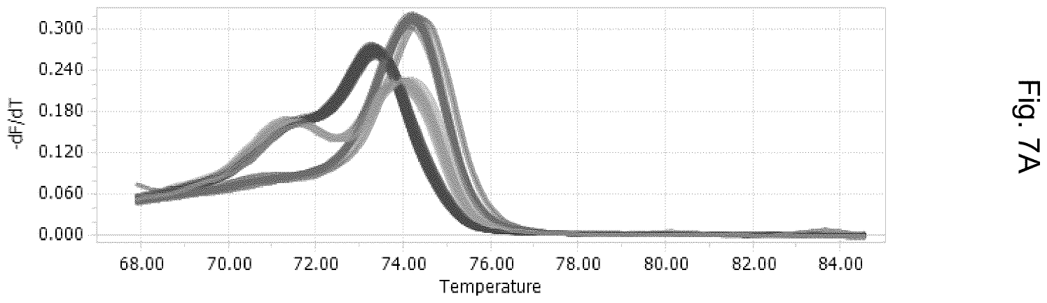

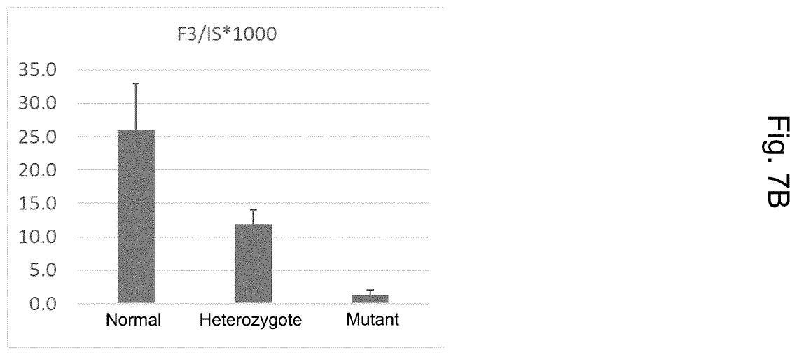

Mutant 3 (also referred to as “16SN693”) has substitution from C to T at the 42,717,998th nucleotides of soybean chromosome 20. Primers for PCR amplification targeting this mutation (forward primer: AGAAAAATCTCTCTCCTGATTGAAA (SEQ ID NO:10) and reverse primer: AGTAATAAGATAGCAATCAAAGGCA (SEQ ID NO:11)) were designed to detect the nucleotide polymorphisms contained in the PCR fragments by the difference in the melting temperature of the PCR fragments. PCR fragments having the normal allele C show a higher melting temperature than PCR fragments containing the mutant allele T (FIG. 7A). The relationship between the genotype of the DNA marker and the furan fatty acid content was studied using 32 individuals from the F2 population obtained by crossing Fukuyutaka with Mutant 3 as described above. As a result, individuals with the mutant genotype expressed low-furan fatty acid content phenotype. Thus, the low-furan fatty acid content phenotype can be explained by this nucleotide polymorphism (FIG. 7B). The explanations for the blue, red, and orange plots in FIG. 5A also apply to those in FIG. 7A.

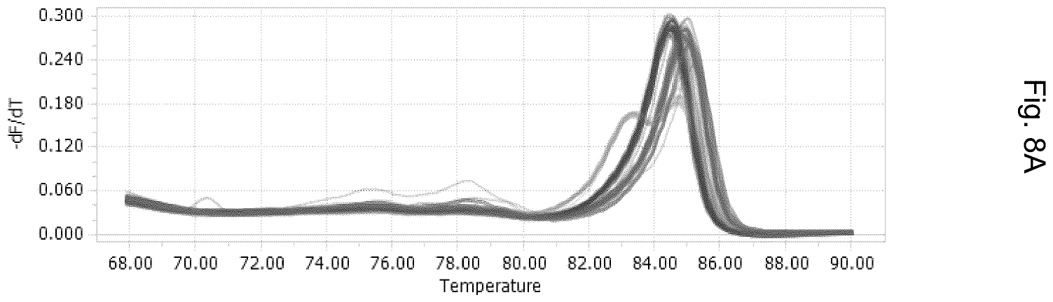

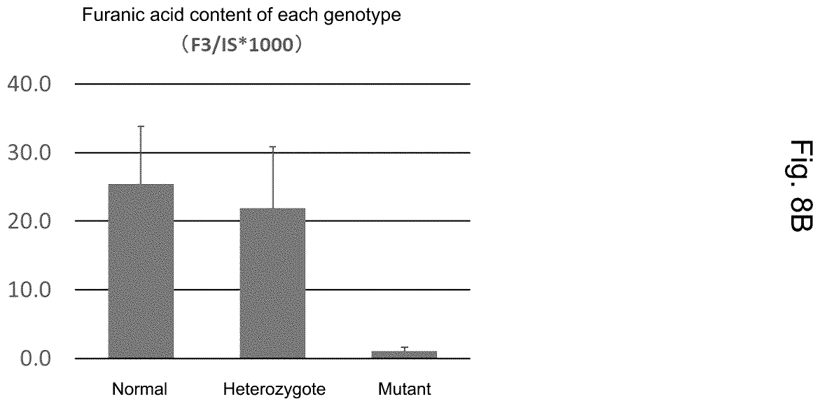

Primers for detecting the DNA polymorphism from G to A at the 4,340,379th nucleotides in soybean chromosome 4 of Mutant 4 (also referred to as “16SN279”) by PCR (forward primer: TCATCACCAGGAGACTCACG (SEQ ID NO: 12) and reverse primer: AAGTTGGGAGGAGGGTTTG (SEQ ID NO:13)) were designed to detect the nucleotide polymorphisms contained in the PCR fragments by the difference in melting temperature of the PCR fragments. If the PCR fragment has the normal nucleotide G, the melting temperature of the PCR fragment will be higher than that of the PCR fragment with the mutant nucleotide A (FIG. 8A). Using this difference in the melting temperature, the relationship between the genotypes of 63 individuals from the F3 population obtained by crossing Fukuyutaka with Mutant 4 and the furan fatty acid content was studied. As a result, individuals having the mutant gene expressed low-furan fatty acid content phenotype. Thus, the low-furan fatty acid content phenotype can be explained by this DNA polymorphism (FIG. 8B). The explanations for the blue, red, and orange plots in FIG. 5A also apply to those in FIG. 8A.

Based on the above results, Mutant lines 1˜4 shown in this example were found to have a reduced furan fatty acid content in the oils/fats contained in the soybeans compared to the original variety Fukuyutaka, showing that the mutations caused in the genes that are thought to encode Cyclopropane fatty-acyl-phospholipid synthase and related methyltransferases, i.e., the Glyma20g34340 and Glyma04g05690 genes, were responsible for the reduced furan fatty acid content exhibited by each mutant line.

### Example 2

<Evaluations of Oils/Fats Derived from Low-Furan Fatty Acid Soybeans and Processed Food Products Using Said Soybeans>

### 1. Extraction and Refinement of Soybean Oil/Fat

### (1) Soybean Seeds

The following seeds grown and harvested in the field at Saga University were used.
1. Fukuyutaka
2. #65 (low-furan fatty acid line): Mutant 1 produced in Example 1

### (2) Extraction Conditions

Each soybean seed, which had been ground using a blender, was immersed in hexane heated to 50° C. and hexane was distilled and collected from the resulting immersion solution to extract the oil content.

### (3) Refinement Conditions

The above extracted oil was refined by degumming, deacidifying, bleaching, and deodorizing, which are common refinement methods.

First, 0.4% by mass of 85% aqueous phosphoric acid solution and 2.5% by mass of warm water were added to the extracted oil heated to 80° C. After stirring for 30 minutes, the aqueous phase was centrifugally removed to obtain degummed oil.

Next, 0.05% by mass of 85% aqueous phosphoric acid solution and 0.6% by mass of 15% aqueous NaOH solution were added to the degummed oil heated to 80° C. After stirring for 15 minutes, the aqueous phase was centrifugally removed. To the resulting oil phase, 15% by mass of ion-exchanged water was added at 80° C. After stirring for 15 minutes, the aqueous phase was centrifugally removed again to obtain deacidified oil.

Next, 1.5% by mass of activated clay was added to the deacidified oil obtained above at 80° C. After stirring under reduced pressure for 30 minutes, the clay was removed by filtration to obtain bleached oil.

Next, the bleached oil obtained above was deodorized by blowing 2.0% by mass of water vapor at 250° C. under reduced pressure for 45 minutes, and 25 ppm by mass of citric acid was added to the treated oil to obtain deodorized oil.

### 2. Evaluation of Soybean Oils/Fats

The deodorized oil derived from each soybean seed and refined as described in section 1 above was used to evaluate the following items (1)-(3).

### (1) General Properties

Fatty acid composition: Measurements were performed in accordance with Standard Methods for the Analysis of Fats, Oils and Related Materials 2.4.2.3-2013.

Furan fatty acid (F3): Measurements were performed according to the method described in paragraphs [0055]-[0058] of Japanese Patent No. 6714001 (or WO2017/033674).

### (2) Light Resistance

Five grams of deodorized oil was placed in a 10 ml vial, and irradiated at 1,000 lux for one week to evaluate the following items. The furan fatty acid content of each deodorized oil was measured before light irradiation.

POV: Measurements were performed in accordance with Standard Methods for the Analysis of Fats, Oils and Related Materials 2.5.2.2-2013.

Light-induced odor: The samples irradiated with light were heated to 100° C. in a block heater and then evaluated for light-induced odor. (n=2)

### (3) Heat Resistance

Hundred grams of oil was poured into a 300 mL beaker and heated at 180° C. (sampling was performed every 5 hours while heating for a total of 20 hours).

Acid value: Measurements were performed in accordance with Standard Methods for the Analysis of Fats, Oils and Related Materials 2.3.1-2013.

Color tone: Measurements were performed using a Lovibond automatic colorimeter in accordance with AOCS Cc13j-97.

Viscosity: Measurements were performed at 30° C. using an E-type viscometer.

Heat-induced odor: For the oil after 15 hours of heating, 14 panel members rated each evaluation item on a scale of 0-5 (see below), with the average value being the evaluation value.

### Rating Score:

5: Sensed very strongly
4: Sensed strongly
3: Sensed strongly to some degree
2: Sensed to some degree
1: Did not sense much
0: Not sensed

### (4) Results

The results of each of the above evaluations (1)-(3) are shown in Tables 1-7.

TABLE 1
Fatty acid composition (mass %)

| | C16:0 | C18:0 | C18:1 | C18:2 | C18:3 | C20:0 | C22:0 | C24:0 | Other |
|---|---|---|---|---|---|---|---|---|---|
| Fukuyutaka | 10.1 | 3.0 | 22.8 | 55.8 | 6.8 | 0.3 | 0.4 | 0.2 | 0.6 |
| #65 | 10.6 | 3.4 | 22.8 | 55.4 | 6.1 | 0.3 | 0.4 | 0.2 | 0.9 |

TABLE 2
Furan fatty acid (F3, ppm)

| | Furan fatty acid (F3) |
|---|---|
| Fukuyutaka | 116.0 |
| #65 | Below detection limit |

TABLE 3
Results of light resistance evaluation

| | POV (meq/kg) | Light-induced odor |
|---|---|---|
| Fukuyutaka | 2.1 | Light-induced odor was |
| | | sensed |
| #65 | 2.3 | Light-induced odor was |
| | | hardly sensed |

TABLE 4
Results of heat resistance evaluation (color tone, 1 inch cell)

| | | 0 | 5 | 10 | 15 | 20 | 25 |
|---|---|---|---|---|---|---|---|
| | | hr | hr | hr | hr | hr | hr |
| Fukuyutaka | 10R + Y | 0.5 | 1.3 | 3.2 | 7 | 14.4 | 20 |
| | Rate of | 0 | 160 | 540 | 1,300 | 2,780 | 3,900 |
| | increase | | | | | | |
| | (%) | | | | | | |
| #65 | 10R + Y | 0.6 | 0.9 | 1.4 | 3.1 | 6.9 | 10 |
| | Rate of | 0 | 50 | 133 | 417 | 1,050 | 1,567 |
| | increase | | | | | | |
| | (%) | | | | | | |

TABLE 5
Results of heat resistance evaluation (acid value)

| | | 0 hr | 5 hr | 10 hr | 15 hr | 20 hr | 25 hr |
|---|---|---|---|---|---|---|---|
| Fukuyutaka | Acid value (mg/g) | 0.05 | 0.07 | 0.10 | 0.21 | 0.39 | 0.53 |
| | Rate of increase (%) | 0 | 50 | 114 | 342 | 703 | 993 |
| #65 | Acid value (mg/g) | 0.06 | 0.06 | 0.08 | 0.11 | 0.16 | 0.23 |
| | Rate of increase (%) | 0 | 6 | 30 | 82 | 173 | 287 |

TABLE 6
Results of heat resistance evaluation (viscosity)

| | | 0 | 5 | 10 | 15 | 20 | 25 |
|---|---|---|---|---|---|---|---|
| | | hr | hr | hr | hr | hr | hr |
| Fukuyutaka | Viscosity | 40.96 | 44.83 | 51.08 | 65.7 | 92.8 | 120.3 |
| | (mPa · s) | | | | | | |
| | Rate of | 0 | 9 | 25 | 60 | 127 | 194 |
| | increase (%) | | | | | | |
| #65 | Viscosity | 41.94 | 44.23 | 46.67 | 52.03 | 60.22 | 68.8 |
| | (mPa · s) | | | | | | |
| | Rate of | 0 | 5 | 11 | 24 | 44 | 64 |
| | increase (%) | | | | | | |

TABLE 7
Results of heat resistance evaluation (heat-induced odor)

| | Fukuyutaka | #65 |
|---|---|---|
| Overall Smell | 3.32 | 1.91 |
| Irritating odor | 2.45 | 1.36 |
| Rancid odor | 1.41 | 0.59 |
| Green off-odor | 0.82 | 0.50 |
| Metal-like smell | 0.32 | 0.18 |
| Paint-like smell | 0.27 | 0.09 |
| Fish-like smell | 0.45 | 0.36 |
| Sweet smell | 0.30 | 0.23 |

Furan fatty acid (F3) in the soybean oil/fat collected from #65 was below the detection limit. The soybean oil/fat collected from #65 had superior light resistance and suppressed light-induced odor compared to the soybean oil/fat collected from Fukuyutaka and else. In addition, it also had excellent heat resistance, the increases in the color tone, oxidation, and viscosity caused during heating were all suppressed, and the heat-induced odor was also suppressed.

### 3. Evaluation of Processed Food Products Using Soybean

(1) Each soybean (Fukuyutaka, #65 (low-furan fatty acid line)) used in section 1. above was used to make prototypes of the following processed food products (i)-(v).
(i) Soybean hamburger steak
(ii) Soy milk
(iii) Bean curd
(iv) Soy pulp cookies
(iv) Soy flour cookies

### (2) Methods of Producing Prototypes

### (i) Soybean Hamburger Steak

One hundred and fifty grams of each soybean was soaked in at least 4 times the amount of water overnight and boiled in a pot for 30 minutes to 1 hour.

Two hundred grams of boiled soybean was made into a paste using a food processor and kneaded well with the ingredients marked with * in Table 8.

The resultant was shaped and fried in an oiled pan.

After 3.5 minutes, the hamburger steak was turned over and fried for another 2.5 minutes to obtain a soybean hamburger steak.

TABLE 8
Ingredients of soybean hamburger

| Ingredients | Weight | |
|---|---|---|
| Boiled soybean | 200 | g |
| *Egg | 25 | g |
| *Breadcrumbs | 7 | g |
| *Potato starch | 20 | g |
| *Salt | 2.4 | g |
| Commercially available deodorized soybean oil | 14 | g |

(“Rich and tasty soybean oil (Koku to umami no
daizu no abura)”, manufactured by J-OIL MILLS)

### (ii) Soy Milk

One hundred and fifty grams of each soybean was soaked in water.

The soybean was lightly rinsed with water, to which 750 g of water (5 times the amount of soybean in dry state) was added, and the resultant was ground with a mixer.

The mixture was heated to boil in a pot and further heated on low heat for another 10 minutes.

The mixture was strained through a bleached cotton cloth to separate the soy pulp from the soymilk to obtain soymilk.

(iii) Bean Curd

Soy milk derived from each soybean in (ii) above was placed in a pot, heated to 75-80° C., and then the heat was turned off.

Bittern was added at 1.5% by mass to the soy milk, and the mixture was lightly stirred, covered with a lid, and steeped for 10 minutes.

Water was drained in a colander covered with a bleached cotton cloth.

### (iv) Soy Pulp Cookies

The water content was squeezed out well of the soy pulp derived from each soybean that was separated from the soy milk in (ii) above, thereby obtaining raw soy pulp.

The ingredients listed in Table 9 were mixed well in a bowl.

The mixture was left to sit in the refrigerator for an hour, then shaped.

The resultant was baked in an oven preheated to 170° C. for 25 minutes to obtain soy pulp cookies.

TABLE 9
Ingredients of soy pulp cookies

| Ingredients | Weight |
|---|---|
| Raw soy pulp | 60 g |
| Weak flour | 40 g |
| Sugar | 15 g |
| Egg | 40 g |
| Butter | 40 g |

### (v) Soy Flour Cookies

Each soybean was ground with a mixer to obtain soy flour.

The ingredients listed in Table 10 were mixed well in a bowl.

The mixture was spread thinly and let sit in the refrigerator for an hour.

After cutting out the shapes, the resultant was baked in an oven preheated to 170° C. for 20 minutes.

TABLE 10
Ingredients of soy flour cookies

| Ingredients | Weight |
|---|---|
| Soy flour | 60 g |
| Weak flour | 40 g |
| Sugar | 15 g |
| Egg | 40 g |
| Butter | 40 g |

### (3) Evaluation Results

For each processed food, a panel of four people rated the “soybeaniness (beany smell)” and “astringent aftertaste (astringency)” on a scale of 1-3 (see below), with the average value being the evaluation value. The results of the evaluation are shown in Tables 11-15.

### Rating Score:

1: Not sensed at all
2: Sensed to some degree
3: Clearly sensed

TABLE 11
Flavor of soybean hamburger steak

| | Fukuyutaka | #65 |
|---|---|---|
| Beany smell | 3 | 2 |
| Astringency | 3 | 1.3 |

TABLE 12
Flavor of soy milk

| | Fukuyutaka | #65 |
|---|---|---|
| Beany smell | 3 | 1.3 |
| Astringency | 2 | 1 |

TABLE 13
Flavor of bean curd

| | Fukuyutaka | #65 |
|---|---|---|
| Beany smell | 3 | 1.8 |
| Astringency | 3 | 1 |

TABLE 14
Flavor of soy pulp cookies

| | Fukuyutaka | #65 |
|---|---|---|
| Beany smell | 3 | 1 |
| Astringency | 2 | 1 |

TABLE 15
Flavor of soy flour cookies

| | Fukuyutaka | #65 |
|---|---|---|
| Beany smell | 3 | 1.3 |
| Astringency | 2.5 | 1 |

Processed food products made using #65 or a processed product of #65 soybean as an ingredient had a good flavor with both beany smell and astringency being suppressed compared to processed food products that were made using Fukuyutaka or a processed product of Fukuyutaka soybean as an ingredient.

### INDUSTRIAL APPLICABILITY

The present invention can provide a modified soybean with reduced furan fatty acid content (production amount of furan fatty acid) in the oil/fat contained in the soybean. An edible oil/fat derived from this modified soybean as well as food products containing this edible oil/fat are different from those containing conventional soybean oils/fats in that the unpleasant light-induced odor peculiar to soybean oils/fats is suppressed, and that the heat-induced odor caused during heat cooking, etc., especially the irritating odor derived from soybean oils/fats, is suppressed. Furthermore, in the edible oil/fat derived from the modified soybean, the increases in viscosity, color tone and oxidation caused during heating are more suppressed than those in conventional soybean oils/fats. In addition, food products and the like containing the modified soybean or a processed product of said modified soybean have a good flavor with a suppressed beany smell. Therefore, the above modified soybean is highly useful and practical.

### SEQUENCE LISTING FREE TEXT

SEQ ID NOS:6-13: Synthetic DNA

1. A modified soybean in which the content ratio of furan fatty acid in the oil/fat contained in the soybean has been reduced by genetic mutation.

2. The soybean according to claim 1, wherein the content ratio is 0 ppm by mass or more and 20 ppm by mass or less.

3. The soybean according to claim 1, wherein the furan fatty acid is 3,4-dimethyl-5-pentyl-2-furannonanoic acid.

4. The soybean according to claim 1, which has a mutation introduced into Glyma20g34340 gene and/or Glyma04g05690 gene.

5. The soybean according to claim 4, wherein the mutation introduced into the gene is any of the mutations selected from the group consisting of missense mutation, nonsense mutation, frameshift mutation, and null mutation.

6. The soybean according to claim 4, wherein the mutation introduced into the Glyma20g34340 gene is:
(i) a mutation of a nucleotide in the codon encoding the 410th amino acid residue of the protein encoded by the gene;
(ii) a mutation of a nucleotide in the codon encoding the 688th amino acid residue of the protein encoded by the gene; or
(iii) a mutation of a nucleotide present at the boundary between the 17th exon and intron of the gene.

7. The soybean according to claim 6, wherein the mutation of (i) above is a mutation where a codon of glycine is replaced with a codon of aspartic acid.

8. The soybean according to claim 7, wherein the mutation of (i) above is a mutation where the 1519th nucleotide, G (guanine), in the cDNA of the Glyma20g34340 gene is replaced with A (adenine).

9. The soybean according to claim 6, wherein the mutation of (ii) above is a mutation where a codon of aspartic acid is replaced with a codon of asparagine.

10. The soybean according to claim 9, wherein the mutation of (ii) above is a mutation where the 2352nd nucleotide, G (guanine), in the cDNA of the Glyma20g34340 gene is replaced with A (adenine).

11. The soybean according to claim 6, wherein the mutation of (iii) above is a mutation where the 7180th nucleotide, G (guanine), in the mRNA precursor of the Glyma20g34340 gene is replaced with A (adenine).

12. The soybean according to claim 4, wherein the mutation introduced into the Glyma04g05690 gene is a mutation of a nucleotide in the codon encoding the 64th amino acid residue of the protein encoded by the gene.

13. The soybean according to claim 12, wherein the mutation is a mutation where a codon of arginine is replaced with a codon of cysteine.

14. The soybean according to claim 13, wherein the mutation is a mutation where the 483rd nucleotide, C (cytosine), in the cDNA of the Glyma04g05690 gene is replaced with T (thymine).

15. The soybean according to claim 1, which is modified Fukuyutaka soybean.

16. LF65 soybean whose accession number is FERM BP-22392.

17. 16SN279 soybean whose accession number is FERM BP-22413.

18. An ingredient for food products, feed products or pharmaceutical compositions, the ingredient comprising the soybean according to claim 1 or a processed product thereof.

19. A food product, feed product or pharmaceutical composition comprising the ingredient according to claim 18.

20. A soybean oil/fat extracted from the soybean according to claim 1.

21. A food product, feed product or pharmaceutical composition comprising the soybean oil/fat according to claim 20.

22. A method for producing a modified soybean, the method comprising crossing the soybeans according to claim 1 with each other, or crossing said soybean with other soybean species.
